(19) United States
(12) Patent Application Publication
(10) Pub. No.: US 2025/0224361 A1
(43) Pub. Date: Jul. 10, 2025

(54) A METHOD OF DETECTING AN ANALYTE IN A SAMPLE AND A BIOSENSOR, A METHOD OF PRODUCING THE BIOSENSOR AND AN ELECTROCHEMICAL SENSOR SYSTEM COMPRISING THE BIOSENSOR

(71) Applicant: Solsten Diagnostics International ApS, Aarhus N (DK)

(72) Inventors: Niels Tækker FOGED, Aarhus N (DK); Eva Maria Nornes OLSEN, Aarhus N (DK); Mark Jonas HAASTRUP, Aarhus N (DK)

(21) Appl. No.: 19/009,538

(22) Filed: Jan. 3, 2025

Related U.S. Application Data
(63) Continuation of application No. PCT/DK2023/050178, filed on Jul. 4, 2023.

(30) Foreign Application Priority Data
Jul. 4, 2022 (DK) PA 2022 70359

(51) Int. Cl. G01N 27/327; C12Q 1/6816

(52) U.S. Cl. CPC G01N 27/3276; C12Q 1/6816

(57) ABSTRACT

A method of detecting an analyte in a sample, a versatile and/or relatively low cost electrochemical biosensor, a biosensor system and an electrochemical biosensor production method are disclosed. The biosensor production method comprises immobilizing basis compounds to a working electrode located in a measuring compartment wherein the basis compounds each comprising an electrode-attachment moiety and a primary region selected from a linker region or a capture region. The biosensor with the immobilized basis compound is universal in particular where the primary region is the linker region where the biosensor may be treated with a capture compound to provide that the biosensor together with a reporter reagent may be specific for an analyte. The electrochemical biosensor may be produced at relatively low cost and open up for desirable fast and accurate detection of analytes.

### CROSS-REFERENCE TO RELATED APPLICATIONS

This application is a bypass continuation of International Application No. PCT/DK2023/050178 filed Jul. 4, 2023, and claims the benefit of Denmark Patent Application No. PA 2022 70359, filed Jul. 4, 2022. The contents of these applications are incorporated herein by reference in their entirety.

### TECHNICAL FIELD

The invention relates to quantitative and/or qualitative electrochemical detection of an analyte in a sample particularly with focus on detecting biochemical analytes. The invention relates to a method of detecting an analyte in a sample as well as an electrochemical sensor system, an electrochemical biosensor as well as a desirable biosensor production method.

### BACKGROUND ART

Electrochemical detection systems and methods are widely used for detection of blood glucose and more recently have also been employed for detection of other biochemical analytes, including diagnostic biomarkers. The development of electrochemical detection systems has led to relatively sensitive and low-cost analytical tools for detection of biochemical analytes, including complexes involving single or double stranded oligonucleotides.

The development of rapid, low-cost, and easy-to-perform biosensors with high sensitivity and selectivity are key for portable point-of-care diagnostics or environmental tests. Currently, detection identification and/or quantification of targets are performed by time-consuming (hours-days) traditional laboratory-based methods e.g., enzyme-linked immunosorbent assay (ELISA), Western Blot, and polymerase chain reaction (PCR), which require skillful processing by specialized operators. Emerging methods, to overcome these limitations, based on optical, electrochemical, or piezoelectric transducers have been demonstrated and especially electrochemical biosensors have received a lot of attention as promising platforms for detection of a number of clinically important targets, such as biomarkers in serum, plasma, or urine, microorganisms in seawater and tap water, and drug levels in serum.

Electrochemical biosensors with single stranded DNA (ssDNA) aptamers that specifically interact with a target have recently attracted great interest. They may be miniaturized, used with portable instruments, mass-produced at relatively low cost, and provide a fast analytical response with only a few microliters of sample. While DNA is generally known as the biological macromolecule responsible for the storage of hereditary information, depending on the individual nucleotide sequence and length, ssDNAs can fold into distinct three-dimensional structures capable of binding non-covalently with high affinity and specificity to a target molecule. These aptameric oligonucleotides may be generated and selected via an efficient in vitro process known as the Systematic Evolution of Ligands by Exponential enrichment (SELEX), from which candidates are screened and characterized before use in e.g., biosensors. As these aptamers show much higher stability than antibodies and are easy to synthesize and chemically modify by solid-phase synthesis, they may be used in the biosensor receptor layer as biorecognition elements. This has been demonstrated for detection of proteins such as thrombin.

Current strategies for immobilization of aptamers to an electrode surface involve direct bonding of thiol-modified aptamers or binding of aptamers to short linkers typically assembled by Au—S chemistry on gold surfaces.

WO17062591 describes a biosensor device for the real-time detection of a target analyte, which includes a receptor component operatively connected to a transducer component which is adapted to interpret and transmit a detectable signal. The receptor component includes a sensing element capable of detecting and binding to at least one target analyte, and a self-assembled monolayer (SAM) layer. The SAM layer is positioned between and in contact with the sensing element and an electrode such that the sensing element, in the presence of the target analyte, causes a detectable signal capable of being transmitted to the electrode.

WO07092552 describes a device and methods for the detection and quantification of one or more target agents in a sample by rapid and specific electrochemical detection. The method includes mixing the sample suspected of containing the target agent with a capture-associated universal oligonucleotide conjugated to a capture moiety to allow the capture moiety to bind the target agent to form a mixture. The mixture is then contacted with immobilized binding partners specific for a capture moiety that has not bound a target agent (i.e., an “unreacted capture moiety”). The unreacted capture moiety can react with (e.g., bind to or otherwise associate with) the immobilized binding partners, thereby immobilizing capture-associated universal oligonucleotides that are conjugated to unreacted capture moieties (“unreacted capture-associated universal oligonucleotides”) from solution. The resultant solution is then contacted with the electrodeassociated universal oligonucleotide, where a hybridization event between the electrode-associated universal oligonucleotide and the capture-associated universal oligonucleotide indicates that a target agent was present in the sample.

Some prior art electrochemical systems use aptamers for selective detection. U.S. Pat. No. 11,172,339 describes such a platform using printed working electrodes functionalized with aptamer for detecting target. Impedance changes as measured as target binds to electrode.

WO15149184 describes a target detection and quantification systems and method based on the use of steric hindrance, either created by the target itself or a macromolecular entity used to bind to the target, to prevent or limit the hybridizing between an anchoring oligonucleotide and a signaling oligonucleotide or a combination of signaling oligonucleotides capable of specifically binding the target or the macromolecular entity.

### SUMMARY OF THE INVENTION

An object of the present invention is to provide an electrochemical method which has a high sensitivity and which may be applied for a wide range of analytes.

In an embodiment, it is an object to provide an electrochemical method which is relatively fast and which simultaneously is highly reliable.

In an embodiment, it is an object to provide an electrochemical sensor system which is relatively simple and fast to use.

In an embodiment, it is an object to provide an electrochemical sensor system comprising an electrochemical biosensor which may be universal—i.e. it may be used for selective detection of several different analytes.

In an embodiment, it is an object to provide an electrochemical sensor system, where the risk of non-specific adherence or binding to the working electrode may be very low or even fully avoided.

In an embodiment, it is an object to provide an electrochemical sensor system, which may be provided at attractive low cost.

In an embodiment, it is an object to provide a versatile and/or low cost biosensor production method. Such versatile and/or low cost biosensor production method is very desirable and provides a large contribution to the technical field.

These and other objects have been solved by the invention or embodiments thereof as defined in the claims and/or as described herein below.

It has been found that the invention or embodiments thereof have a number of additional advantages, which will be clear to the skilled person from the following description.

The method of detecting an analyte in a sample comprises providing the steps of providing
an electrochemical sensor system comprising an electrochemical biosensor and an electrochemical reader, wherein the electrochemical biosensor comprises a measuring compartment comprising a working electrode (WE), wherein the working electrode comprises immobilized basis compounds, wherein each basis compound comprises an electrode-attachment moiety and a primary region; and
a reporter reagent comprising reporter compounds, wherein each of the reporter compound comprises a capture-binding region and a reporter moiety;

The basis compounds are selected from i) linker compounds wherein the primary region is a linker-region, ii) direct capture compounds wherein the primary region is a capture region or iii) pre-hybridized compounds of linker compounds comprising a linker region and capture compounds comprising a linker-binding region hybridized with the linker region and a capture region that is the primary region.

The inventors have found that by providing the electrochemical biosensor according to the invention a highly reliable and relatively simple electrochemical biosensor is provided which in addition may be produced at relatively low cost.

The inventors have found that by providing the electrochemical biosensor with a basis compound that is a linker compound, which comprises a primary region, that is a linker region configured for acting as a universal linker for immobilizing a capture compound which may have any desired capture region, the electrochemical biosensor may be applied as a universal biosensor. Thereby a highly versatile electrochemical biosensor has been provided.

In addition, the inventors have found that by providing the electrochemical biosensor with a basis compound that is a direct capture compound, which comprises a primary region that may be any desired directly immobilized specific capture region, the electrochemical biosensor may be particularly simple to manufacture. Thereby an electrochemical biosensor may be provided at attractive low cost.

The inventors have also found that by providing the electrochemical biosensor with a basis compound that is a pre-hybridized compound of a linker compound, which comprises a universal linker region, and a capture compound, which comprises a linker-binding region hybridized with the universal linker region and a primary region, that is any desired capture region, the electrochemical biosensor may be uniform and simple to manufacture. Thereby a flexible electrochemical biosensor may be provided at attractive modest cost.

The capture region of the capture compound or the direct capture compound is selected to be capable of binding a selected target, which may be the analyte and/or may be the capture-binding region of the reporter compound as explained further below.

In practice such universal, versatile, simple and/or economical electrochemical biosensors may be fabricated at a central location, where they may be mass produced to have a very high quality and/or be fabricated at a relatively low cost. The manufacturing of the versatile electrochemical biosensor may be optimized as further explained below. The user, or a user supplier may then in a relatively simple way post process the universal electrochemical biosensors to biosensors directed for a specific target (analyte and/or reporter compound) thereby providing that the user may perform a selected assay very fast and with a desired high accuracy as it will be further explained and exemplified below.

The term respective terms “capture region” and “capture-binding region” are herein used to mean regions of respectively the capture compounds or direct capture compounds, and the reporter compounds which are capable of binding to each other at the assay conditions.

The respective capture region and the capture-binding region may comprise any moieties capable of forming the mutual binding, such as nucleotides, stretches of nucleotides or a portion of a stretch of nucleotides or a region of nucleotides.

The phrase “stretch of nucleotides” means herein a sequence of at least two nucleotides in a row.

The phrase “region of nucleotides” means herein a region of a compound comprising nucleotides.

It should be emphasized that the term “comprises/comprising” when used herein is to be interpreted as an open term, i.e. it should be taken to specify the presence of specifically stated feature(s), such as element(s), unit(s), integer(s), step(s) component(s) and combination(s) thereof, but does not preclude the presence or addition of one or more other features.

Throughout the description or claims, the singular encompasses the plural, and the plural encompasses the singular unless otherwise specified or required by the context.

The “an embodiment” should be interpreted to include examples of the invention comprising the feature(s) of the mentioned embodiment.

The term “substantially” should herein be taken to mean that ordinary product variances and tolerances are comprised. All features of the invention and embodiments of the invention as described herein, including ranges and preferred ranges, may be combined in various ways within the scope of the invention, unless there are specific reasons not to combine such features.

Unless other is specified, any properties, ranges of properties and/or determination and/or assay condition is given or provided at 1 atmosphere and 25° C.

All features of the invention and embodiments of the invention as described herein including ranges and preferred ranges may be combined in various ways within the scope of the invention, unless there are specific reasons not to combine such features.

To provide an effective assay where the basis compounds are direct capture compounds, each of the primary region in the form of the capture region and the capture-binding region comprises an oligonucleotide. The capture region and the capture-binding region are selected such that the capture-binding region is adapted for hybridizing with the capture region.

Where the basis compounds are not direct capture compounds, each of the primary region, linker region, linker-binding region, capture region and capture-binding region comprises an oligonucleotide in order to ensure an effective assay. The linker region and the linker-binding region are selected such that the linker-binding region is adapted for hybridizing with the linker region. The capture region and the capture-binding region are selected such that the capture-binding region is adapted for hybridizing with the capture region.

In addition, one of the capture region and the capture-binding region comprises an aptamer for the analyte, and at least part of the aptamer contributes to the hybridization of the capture region and the capture-binding region.

The other one of the capture region and the capture-binding region, preferably does not comprise an aptamer.

The capture reagent and the reporter reagent may independently of each other comprise a suitable buffer, such as a DPBS buffer optionally comprising MgCl₂, such as 5 mM MgCl₂. Mg₂⁺ may act as a stabilizer for stabilizing cationic loaded oligonucleotides.

In an embodiment, the buffer is or comprises one of the twenty Good's buffers or a combination comprising one or more of the good's buffers. Advantageously, the buffer is or comprises a zwitterionic sulfonic acid buffering agent, such as HEPES (4-(2-hydroxyethyl)-1-piperazineethanesulfonic acid).

Advantageously, any of the reagents as described herein and independently of each other, such as one or more of the reporter reagent, the antifouling reagent, the basic reagent, the linker reagent, and/or the capture reagent, comprises a buffer suitable for the compound(s) of the reagent. The buffer may in principle be any buffer that is not reacting with the compound(s) of the reagent. Preferably the buffer is selected from buffers comprising one or more of citrate buffer, sodium hydroxide buffer, potassium hydroxide buffer, PBS buffer and/or one or more of the buffers of Good's buffers or any modifications thereof. Such Good's buffers are available from for example Merck.

Preferred buffers includes buffers comprising one or more of the Good's buffers, PBS, DPBS, HEPES, Tris, or any other Good's buffers optionally comprising MgCl₂ or CaCl₂, preferably from 1 to 20 nM, more preferably from 2 to 10 nM; and/or comprise antifouling agents as described herein such as albumin up to 5% w/v, such as 0.5% w/v.

The method further comprises
applying a portion of the capture reagent to the measuring compartment and allowing the linker region to hybridize with the linker-binding region, where the basis compounds are linker compounds,
subjecting a portion of the reporter reagent to the sample and to the measuring compartment and detecting the analyte, comprising read-out of at least one electrical signal using the reader and
quantitatively and/or qualitatively detecting the analyte based on the electrical signal.

Since the electrochemical biosensor may be produced at relatively low cost, it may be suitable for single-use and/or disposable biosensors, as well as suitable for qualitative assays, such a high throughput screening assays and/or infection detection quick tests.

However, it has been found that the electrochemical biosensor and the method of detecting an analyte has high sensitivity and is highly suitable for performing quantitative detection of the analyte.

The reporter moiety may conveniently comprise a redox mediator, such as methylene blue, methylene blue derivatives; ferrocene, ferrocene derivatives; anthraquinone, anthraquinone-2,6-disulfonate and other anthraquinone derivatives, ruthenium hexamine, dimethyl sulfoxide ruthenium and other ruthenium complexes, ferritin derivatives, cobalt, cobalt derivatives, [Co(GA)2(phen)] (GA=glycolic acid, phen=1,10-phenanthroline), metal nanoparticles (e.g., Au, Pt, Pd, Ag, Cu), pyrroloquinoline quinone (PQQ), benzoquine, Osmium (III) complexes, diphenylamine, neutral red, toluidine blue, phenosafranine, oxidoreductases or other enzymes capable of electron transfer-reactions or catalysis of redox reactions or any combination thereof.

When subjecting the portion of the reporter reagent to the sample and to the measuring compartment an electrochemically cell may be formed enabling an electrochemical detection e.g. comprising reading at least one electrical signal such as performing at least one potentiometric, amperometric, or impedometric measurement. This will be further described and exemplified below.

The oligonucleotide may be any kind of oligonucleotide natural or synthetic or combinations thereof. The oligonucleotide or one or more nucleotides thereof may be modified. The oligonucleotides may be a stretch of nucleotides or a portion of a stretch of nucleotides or a region of nucleotides.

The oligonucleotides of the primary region, the linker region, the linker-binding region, the capture region and the capture-binding region may for example independently of each other be selected from natural or synthetic or partly synthetic single stranded oligonucleotides, such as DNA oligonucleotides, RNA oligonucleotides, PNA oligonucleotides, LNA oligonucleotides, SNA oligonucleotides, XNA oligonucleotides or a mixture thereof, preferably the linker region or the capture region of the direct capture compound is a PNA oligonucleotide or a DNA oligonucleotide or a mixture thereof.

Advantageously, the linker region of the linker compound, the linker region of the prehybridized compound or the capture region of the direct capture compound is a PNA oligonucleotide or a DNA oligonucleotide or a mixture thereof.

The two natural types of oligonucleotides are DNA and RNA, however, there also exists modified oligonucleotides like PNA, LNA and SNA. Oligonucleotides can form double helices and other structures through base pairing. Preferred oligonucleotides are described below.

PNA has a neutral backbone compared to DNAs negative charged backbone and this is one of the reason for that PNA:DNA hybridization is stronger that DNA:DNA hybridization.

Both DNA and PNA can be synthesized easily in-vitro. For PNA it is easy to synthesize a PNA-peptide conjugate because the PNA backbone consist of amide bonds, and it synthesized by peptide chemistry. PNA oligonucleotides and PNA-peptides are commercially available.

In an embodiment, the oligonucleotide(s) may comprise nucleic acid analogues referred to as XNA. XNA is for example described in https://en.wikipedia.org/wiki/Xeno_nucleic_acid

The oligonucleotides may optionally be modified e.g. as described in https://www.glenresearch.com/browse/labels-and-modifiers

Aptamers are single stranded oligonucleotides (preferably DNA or RNA oligonucleotide) or oligopeptides which are developed to bind to specific targets. A target, such as a substance or chemical constituent that is of interest is also referred to as an analyte. The targets/analytes may for example be small molecules, proteins, disease markers and cells. Aptamers are alternatives to antibodies and can be used in diagnostics to detect and quantify a specific target in a sample.

In 1990, an in-vitro method for selection of oligonucleotide aptamers was discovered independently by two groups Tuerk & Gold (Science, 249, 505-510) and Ellington & Szostak 1990 (Nature, 346, 812-822). The method is called SELEX, Sequential Evolution of Ligands by Exponential Enrichment, and works by an iterative process where the aptamer for a specific target is selected from an oligonucleotide library and for each selection round non-binding aptamers is discarded and the aptamers which bind is replicated before being subjected to another round of selection. The process always includes positive selection rounds and can also be followed by counterselection in subsequent rounds. Finally, the best aptamers may be sequenced by for example Next Generation Sequencing and tested for target affinity.

There are several advantages of aptamers over antibodies. Aptamers are easily synthesized in-vitro and are commercially available. They may be modified with a range of functional groups and molecules and there is no batch-to-batch variation. Also, aptamers have a high thermal and chemical stability. For any oligonucleotide aptamer, it is simple to design and synthesize complementary oligonucleotides with a range of binding affinities.

In an embodiment, the oligonucleotide of the linker region of the linker compound or of the pre-hybridized compound or the capture region of the direct capture compound is a PNA oligonucleotide.

It has been found that a linker compound or a pre-hybridized compound in which the oligonucleotide of the linker region is a PNA is very attractive. Similarly, a direct capture compound in which the capture region is a PNA is very attractive. PNAs are modified oligonucleotides where the purine and pyrimidine bases are linked to the backbone through amide bonds like in peptides. PNA is easily synthesized with high specificity e.g. by solid phase peptide chemistry. PNAs are highly stable molecules, which in the electrochemical biosensor and the production thereof may be very beneficial. In addition, the PNAs may be modified e.g. to provide a spacer domain and/or an antifouling moiety as described further below.

Providing the basis compound to have a PNA linker region or a PNA capture region, may therefore ensure a long shelf time of the electrochemical biosensor.

The basis compound may conveniently be immobilized to the working electrode via the electrode-attachment moiety as further described below in the description of the electrochemical biosensor production.

The electrode-attachment moiety may advantageously be selected in dependence of the working electrode material and may comprise any suitable moiety capable of chemically immobilizing the basis compound to the surface of the working electrode.

In an embodiment, the basis compounds are immobilized using Click chemistry and the electrode-attachment moiety is a moiety forming part of the click attachment. Click chemistry is well known in the art and an example is described in US209352785.

In an embodiment, the electrode-attachment moiety is a silane coupling moiety.

Examples of suitable electrode-attachment moieties includes an amine moiety suitable for coupling at least one of N, N′-dicyclohexylcarbodiimide (DCC), 1-ethyl-3-(3-dimethylaminopropyl) carbodiimide (EDC), N-hydroxysuccinimide (NHS) and/or sulfo-NHS carbonyldiimidazol.

Advantageously, the electrode-attachment moiety comprises at least one thiol group, such as 2-5 thiol groups, e.g. 3 thiol groups. The attachment moiety may advantageously comprise one or more cysteine amino acids comprising the thiol group(s). Conjugation of one or more cysteine amino acids to a PNA oligonucleotide is particularly favourable since the PNA-peptide conjugates can be synthesized by solid phase synthesis.

Using 2 or more thiol groups for the immobilization of the linker compound has been found to provide a very stable and durable immobilization, which in addition may provide a fast immobilization procedure which adds to improve the manufacturing of the electrochemical biosensor.

To reduce non-specific binding for example of nucleic acids, proteins, cell fragments or other components which may disturb the electrochemical detection of the analyte one or more antifouling strategies may be provided as explained below.

It has been found that non-specific binding may be highly reduced or even eliminated by providing the basis compound to have one or more antifouling moieties. This is herein referred to as a first antifouling strategy.

In an embodiment, the basis compound comprises at least one antifouling moiety. The antifouling moiety may form part of the basis compound backbone or may form part of a sidechain.

Examples of desired antifouling moiety includes a zwitterionic moiety or a PEG (poly(ethylene glycol)) based moiety. Preferably the one or more antifouling moieties comprises an oligopeptide, wherein the at least one antifouling moiety comprises cationic and anionic amino acids, such as at least one of lysine, arginine, aspartic acid and glutamic acid, and preferably the antifouling moiety or moieties has/have a molar weight of up to 2000 g/mol.

The antifouling moiety is advantageously located closer to the electrode-attachment moiety than the primary region. In an embodiment, the antifouling moiety is located between the electrode-attachment moiety and the primary region.

In an embodiment, the antifouling moiety is located oppositely to the primary region—i.e. on the opposite side of the electrode-attachment moiety than the primary region. Thereby it may be located very close to the electrochemical biosensor electrodes.

One method to increase specificity and decrease the amount of non-specific binding on biosensor electrodes comprises to conjugate peptide zwitterions to the basis compound.

Zwitterions may comprise of an equal or near equal amount of positive and negative charges.

Lysine and arginine may be applied to introduce a positive charge and aspartic and glutamic acid can introduce a negative charge. Different lengths from 8 to 20 amino acids have been applied and the negative and positive charge can be alternated in groups of 1-2-3 and 4 amino acids.

Examples comprises:
EKEKEKEK
EEKKEEKK
EEEKKKEEEKKK
EEEEKKKKEEEEKKKK

Wherein E represents a positive charged amino acid and K represents a negative amino acid.

It has been found that a second antifouling strategy may comprise providing one or more electrodes, preferably comprising the working electrode of the electrochemical biosensor with an individually immobilized antifouling reagent.

In an embodiment, the electrochemical biosensor therefor further comprises antifouling reagents immobilized individually to the working electrode and optional other electrodes of the electrochemical biosensor. Preferably the antifouling reagents comprises PEG poly(ethylene glycol) (PEG), zwitterions, alkanethiols such as mercaptohexanol (MCH), functionalized n-alkanethiols such as mercaptoproprionic acid (MPA), 11-mercaptoundecanoic acid (MUA), 1-tetradecanethiol (TDT), proteins such as albumin, and/or any combinations comprising one or more of these reagents.

The antifouling reagents has the function of reducing or fully avoiding non-specific binding to the electrode, such as non-specific binding of matrix molecules, macromolecules and/or other sample molecules. Preferably the antifouling reagent may be added to saturate the electrode. The treatment of the working electrode with the antifouling reagent may be provided after and/or simultaneously with immobilizing the basis compounds and/or after application or simultaneously with applying the capture reagent to the measuring compartment.

The antifouling reagent may be immobilized via peptides which may be immobilized through a thiol of a cysteine residue and/or neutral spacer amino acids may be incorporated between cysteine and a zwitterionic sequence.

The first and second antifouling strategies may be applied separately or in combination or in combination with any other suitable antifouling strategies, such as suitable prior art strategies.

For example, it is well known in the art to treat an electrode for reducing biofouling of the electrode in the form of non-specific binding. Example of suitable methods of reducing biofouling are disclosed in Campuzano, Susana et al. “Antifouling (Bio)materials for Electrochemical (Bio)sensing.” International journal of molecular sciences vol. 20,2 423. 19 Jan. 2019, doi:10.3390/ijms20020423

In an embodiment, the basis compound comprises a spacer domain located between the electrode-attachment moiety and the primary region. The spacer domain may have any length, however a relative short spacer domain may be preferred. In an embodiment, the spacer domain comprises a repeating sequence of ethylene glycol units such as triethylene glycol spacer (spacer-9), n-carbon atom spacer, an AEEA spacer, or a sequence of N nucleotides wherein N is an integer from 1 to 10, and/or any combinations comprising one or more of these spacers.

The spacer may be introduced during solid phase synthesis and may be introduced at any desired position. In an embodiment the spacer is introduced between the electrode-attachment moiety and the primary region. This is specifically preferred where the oligonucleotide of the linker region of the linker compound or of the pre-hybridized compound or of the capture region of the direct capture compound is a PNA oligonucleotide or a DNA oligonucleotide.

The spacer may be selected in dependence of the type of linker or capture oligonucleotides. For example for DNA oligonucleotides a spacer-9 may be preferred and for PNA oligonucleotides an AEEA spacer (2-aminoethoxy-2-ethoxy acetic acid) may be preferred.

Further information about suitable spacers for providing the spacer domain may be found here: https://www.pepscan.com/custom-peptide-synthesis/peptide-modifications/linkers-spacers/

Where the basis compound is a linker compound or a pre-hybridized compound, the oligonucleotide of the linker region may comprise n1 nucleotides and the oligonucleotide of the linker-binding region may comprise n2 nucleotides. n1 and n2 are integers which may be equal or differs from each other.

A number N of the n1 nucleotides of the linker region are complementary with a number N of the n2 nucleotides of the linker-binding region. The number n1 of nucleotides of the linker region and the number n2 of the nucleotides of the linker-binding region may in principle and independent of each be any number preferably at least 6.

To ensure that the linker compound is configured for providing or allowing the reporter moiety of the reporter compound to be located relatively close to the working electrode of the electrochemical biosensor upon or after hybridizing between the capture region and the capture-binding region, it is desired that the numbers n1 and n2 independently of each other are not too large such as 100 or less, such as 80 or less.

In an embodiment, the oligonucleotide of the linker region comprises N nucleotides and the oligonucleotide of the linker-binding region comprises N nucleotides wherein the N nucleotides of the linker-binding region and the N nucleotides of the linker region are complementary and wherein N is an integer of at least 6, preferably N is from 10 to 50, more preferably from 14-18.

In an embodiment, the linker region and/or the linker-binding region consists of the respective N complementary oligonucleotides.

It should be noted that the N nucleotides may or may not be a stretch of nucleotides.

Advantageously, the N nucleotides of the oligonucleotide of the linker-binding region and the N nucleotides of the oligonucleotide of the linker region are adapted for hybridizing with each other.

In an embodiment, n1≥18, n2≥18 and N≤16, for example n1=40, n2=20 and N=12.

In an embodiment, n1=N and n2≥N.

In an embodiment, n2=N and n1≥N.

N may beneficially be relatively small because this may facilitate a very fast hybridizing between linker region and the linker-binding region. On the other hand it is also desired that N is sufficient large to facilitate a stable hybridization. Thus N may preferably be from 10 to 18.

The capture compound may advantageously comprise a spacer domain located between the linker-binding region and the capture region. The spacer domain preferably comprises at least one nucleotide, such as from 2 to 10 nucleotides, such as from 3 to 6 nucleotides. Using a relative short spacer may be beneficial to ensure that the reporter moiety of the reporter compound upon hybridizing between the capture region and the capture-binding region may be located relatively close to the working electrode of the electrochemical biosensor and thereby provide a desired high electrical signal.

The spacer domain may be introduced using solid phase synthesis and the introduced spacer nucleotides may or may not be chemically modified.

The nucleotide(s) of the spacer domain may include any nucleotides not forming part of the linker-binding region or the capture region. Thereby the spacer domain remains a single strand region when the linker-binding region is hybridized with the linker region and the capture region is hybridized with a capture-binding region of a reporter compound or is binding an analyte. The spacer domain may for example comprise a stretch of identical nucleotides, such as an AAAAAA stretch or an AAA stretch.

In an embodiment, the capture compound comprises a strand of nucleotides comprising the linker-binding region and the capture region separated by the spacer domain. The spacer domain ensures a high flexibility of the capture region, which may ensure a reduced risk of sterically hindrance in particular where the capture region comprises an aptamer.

The oligonucleotide of the capture region may comprise m1 nucleotides and the oligonucleotide of the capture-binding region may comprise m2 nucleotides. m1 and m2 are integers which may be equal or differs from each other.

A number M of the m1 nucleotides of the capture region are complementary with a number M of the m2 nucleotides of the capture-binding region. The number m1 of nucleotides of the linker region and the number m2 of the nucleotides of the linker-binding region may in principle and independent of each other be any number preferably at least 8.

In an embodiment, the capture region and the capture-binding region independently of each other comprises from 8 to 100, nucleotides, wherein up to 30 nucleotides of the capture-binding region is adapted for hybridizing with complementary nucleotides of the capture region, preferably from 10 to 14 nucleotides of the capture-binding region is adapted for hybridizing with complementary nucleotides of the capture region.

In an embodiment, the oligonucleotide of the capture region comprises M nucleotides and the oligonucleotide of the capture-binding region comprises M nucleotides wherein the M nucleotides of the capture-binding region and the M nucleotides of the capture region are complementary and wherein M is an integer of at least 8, preferably M is from 10 to 30, more preferably from 10-14.

In an embodiment, one of the capture region and the capture-binding region consists of M nucleotides and the other one, preferably in the form of an aptamer comprises more than M nucleotides, i.e. either m1 or m2 is larger than M.

It should be noted that the M nucleotides may or may not be a stretch of nucleotides.

Advantageously, the M nucleotides of the oligonucleotide of the capture-binding region and the M nucleotides of the oligonucleotide of the capture region are adapted for hybridizing with each other.

As described above one of the capture region and the capture-binding region comprises an aptamer for the analyte. An aptamer may advantageously, comprise or consist of 10 to 100 nucleotides.

Due to the conformational shape of an aptamer (e.g. caused by a secondary and/or a tertiary aptamer structure) the nucleotides which contribute to the hybridizing may or may not be consecutive. Thus, in an embodiment the nucleotides of the aptamer which contribute to the hybridizing may be separated by nucleotides that do not contribute to the hybridizing.

To achieve sufficient specificity and moderate affinity at least 8 (or 10) nucleotides must advantageously contribute to the hybridization.

To avoid a very or even too high affinity between the hybridizing oligonucleotides, it is preferred that less than 17 or even less than 15 nucleotides contributes to the hybridization.

A too high affinity between the hybridizing oligonucleotides may reduce the aptamer's binding of the analyte, “disturb the equilibrium” and/or “outcompete the aptameric binding”.

In a first arrangement of the method, the capture region comprises an aptamer for the analyte, and the portion of reporter reagent and the sample are subjected to the measuring compartment.

In the first arrangement of the method, the method may conveniently comprise a competing step, a blocking step or a displacement step as further described below.

In an embodiment of the first arrangement, the step of subjecting of the reporter reagent and the sample to the measuring compartment comprises a competing step comprising adding the portion of reporter reagent and the sample simultaneously to the measuring compartment and allowing the capture region of the respective capture compounds to hybridize to the capture-binding region of one of the reporter compounds of the portion of reporter reagent or to bind one optional analyte of the sample, optionally the method comprises mixing the portion of reporter reagent and the sample prior to the step of adding the portion of reporter reagent and the sample simultaneously to the measuring compartment.

Thereby the capture-binding region and optional analyte of the sample will be in competition for respectively hybridizing with or being bound by the aptamer of the capture region.

Where no analyte is present in the sample, the highest possible number of reporter compounds will be hybridized via its capture-binding region to the capture region and the number of reporter moieties affecting the electrical signal will be high. As it will be clear from the following description, a similar correlation will be seen in other embodiments.

If the analyte is present in the sample, some of these analytes, depending on the concentration of the analytes in the sample, may bind to aptamers of respective capture region and prevent the capture-binding region to hybridizing to these analyte bound aptamers, thereby the number of reporter moieties affecting the electrical signal will be lower than without the presence of the analyte. As it will be clear from the following description, a similar correlation will be seen in other embodiments.

Thus the higher the concentration of the analyte the less reporter moieties will affect the electrical signal and thereby a quantitative and qualitative determination of the analyte may be obtained. As it will be clear from the following description, a similar correlation will be seen in other embodiments.

The method conveniently comprises providing an incubation period after adding the portion of reporter reagent and the sample to the measuring compartment and prior to read-out of the electrical signal. The incubation period may be as long as desired and preferably the incubation period has a duration of at least 30 second, such as from 1 to 15 minutes. The incubation period may ensure sufficient time for the hybridizing to take place.

In an embodiment, the read-out may be initiated before termination of the incubation period, for example the read-out may be initiated immediately after adding the portion of reporter reagent and the sample simultaneously to the measuring compartment.

The incubation period may for example be determined by the period until the read-out of the signal becomes steady.

In an embodiment of the first arrangement, the step of subjecting of the portion of reporter reagent and the sample to the measuring compartment comprises a blocking step comprising adding the sample to the measuring compartment and allowing optional analyte to bind to aptamers of the respective capture compounds, followed by a step of adding the portion of reporter reagent to the measuring compartment allowing the capture-binding region of respective reporter compounds to hybridize with aptamers that have not bound analyte.

In this embodiment the method comprises allowing the optional analyte to bind to the capture region of capture compound followed by allowing capture-binding region of the reporter compounds to hybridize with capture regions that are not already blocked by the analyte.

The method conveniently comprises providing an incubation period between the step of adding the sample and the step of adding the portion of reporter reagent to the measuring compartment and/or wherein the method comprises providing an incubation period after adding the portion of reporter reagent to the measuring compartment and prior to read-out of the electrical signal. The respective incubation periods may have any lengths. Preferably, the one or more incubation periods each has a duration of at least 30 second, such as from 1 to 15 minutes. In an embodiment, the read-out may be initiated before termination of the incubation period after adding the portion of reporter reagent to the measuring compartment. For example the read-out may be initiated immediately after adding the portion of reporter reagent to the measuring compartment.

The incubation period may for example be determined by the period until the read-out of the signal becomes steady.

In an embodiment of the first arrangement, the step of subjecting of the portion of reporter reagent and the sample to the measuring compartment comprises a step of adding the portion of reporter reagent to the measuring compartment allowing respective capture-binding regions of the reporter compound of the portion of reporter reagent to hybridize to respective capture regions of the capture compounds followed by a displacement step comprising adding the sample to the measuring compartment allowing aptamers of non-hybridized capture regions to bind an optional analyte of the sample, wherein the non-hybridized capture regions allowed to bind an optional analyte of the sample comprises de-hybridized capture regions in which a hybridized capture-binding region has been displaced.

Non-hybridized capture regions includes a) capture regions that have not been hybridized with capture-binding regions plus b) capture regions that have hybridized to with capture-binding region, but which at least partly due to the presence of analytes and to establishing chemical equilibrium are detached from their capture-binding regions to which they initially were hybridized with.

The more non-hybridized capture regions of b), the more will the electrical signal be reduced.

In an embodiment, the read-out is performed before and after the displacement step, thereby providing an increased accuracy in the determination of the analyte.

In this embodiment, the method comprises allowing the capture-binding region of the reporter compounds to hybridize with the capture region of the capture compounds followed by allowing the optional analyte to displace the reporter compounds through binding to the aptamer.

The method conveniently comprises providing an incubation period between the step of adding the portion of reporter reagent to the measuring compartment and the step of adding the sample and/or wherein the method comprises providing an incubation period after adding the sample to the measuring compartment and prior to read-out of the electrical signal. The respective incubation periods may have any lengths. Preferably, the one or more incubation periods independently of each other have a duration of at least 30 second, such as from 1 to 15 minutes.

In a special embodiment, the adding of the reporter reagent may be part of the manufacturing process, thereby taking place days, months or even years before the adding of the sample.

In an embodiment, the read-out may be initiated before termination of the incubation period after adding the sample to the measuring compartment. For example the read-out may be initiated immediately after adding the sample to the measuring compartment.

The incubation period may for example be determined by the period until the read-out of the signal becomes steady.

In an embodiment, the read-out for obtaining a baseline signal may be performed after adding the portion of reporter reagent to the measuring compartment and before the displacement step.

The portion of reporter reagent may advantageously comprise sufficient reporter compounds to saturate the capture region.

To provide a desired high signal in the embodiment, it is desired that reporter reagent comprises a number of reporter compounds that is equal to or larger than the number of capture compounds hybridized to the linker region. Thereby, even a small amount of analyte may be detected. The reporter reagent may advantageously, comprise a number of reporter compounds that is at least 90% of the number of capture compounds hybridized to the linker region and/or at least 90% of the number of immobilized linker compounds.

In an embodiment, it is desired that the reporter compound in the portion of reporter reagent is substantially equal to or in surplus relative to the number of immobilized linker compound. If a smaller amount of reporter compound are applied and the amount of analyte is relatively low an undesired large number of capture regions may be left in unreacted condition, which may not be desired where the concentration of analyte is low.

In an embodiment of the first arrangement, the capture-binding region advantageously has a binding affinity to the capture region which is less than the binding affinity between the aptamer of the capture region of the capture compound and the analyte.

In an embodiment, of the first arrangement wherein the capture region comprises the aptamer for the analyte, and wherein the portion of reporter reagent and the sample are added in distinct steps, the method may advantageously comprise a washing after the step of adding one of the portion of reporter reagent and the sample and before adding the other one of the portion of reporter reagent and the sample. The washing step may provide an increased sensitivity in particular where the method comprises a displacement step after the washing step.

In a second arrangement of the method, the capture-binding region comprises an aptamer for the analyte, and the step of subjection of the portion of reporter reagent to the sample and to the measuring compartment comprises mixing the portion of reporter reagent with the sample and adding the mixture to the measuring compartment.

In an embodiment of the second arrangement, the method comprises providing an incubation period between the step of mixing the portion of reporter reagent with the sample and the step of adding the mixture to the measuring compartment and/or the method comprises providing an incubation period after adding the mixture to the measuring compartment and prior to read-out of the electrical signal. The respective incubation periods may have any lengths. Preferably the one or more incubation periods independently of each other have a duration of at least 1 second, such as from 1 to 15 minutes.

In an embodiment, the read-out may be initiated before termination of the incubation period after adding the mixture to the measuring compartment. For example the read-out may be initiated immediately after adding the mixture to the measuring compartment.

The incubation period may for example be determined by the period until the read-out of the signal becomes steady.

In an embodiment of the second arrangement, the portion of reporter reagent comprises deficit reporter compounds to saturate the capture regions.

To provide a desired high sensitivity in an embodiment of the second arrangement, it is desired that reporter reagent comprises a number of reporter compounds that is equal to or less than the number of capture compounds hybridized to the linker region. Thereby, even a small amount of analyte may be detected.

The reporter reagent may of the second arrangement advantageously, comprise a number of reporter compounds that is less than 90% of the number of capture compounds hybridized to the linker region and/or less than 90% of the number of immobilized linker compounds or less than 90% of the number of immobilized direct capture compounds.

In an embodiment of the second arrangement, it is desired that the reporter compound in the portion of reporter reagent is substantially equal to or less relative to the number of immobilized linker compound or the number of immobilized direct capture compound. If a larger amount of reporter compound is applied and the amount of analyte is relatively low so that only an insignificant amount of the reporter compounds are bound to the analyte, it may not substantially reduce the number of capture-binding regions available to hybridize to the capture regions.

In an embodiment of the second arrangement, the capture-binding region has a binding affinity to the capture region which is less than the binding affinity between the aptamer of the capture-binding region and the analyte.

In embodiments of the second arrangement where the capture-binding region comprises the aptamer for the analyte and where the portion of reporter reagent is mixed with the sample advantageously comprises sufficient reporter compounds to provide a desired electrical signal even where the sample have a relatively high concentration of analyte.

In an embodiment, the portion of reporter reagent mixed with the sample comprises deficit reporter compounds to saturate the capture regions, such as deficit reporter compounds to saturate the capture regions even when the sample does not contain any analyte. The deficit reporter compounds may for example be from 25% to 95% of the reporter compounds sufficient to saturate the capture region, more preferably from 50% to 90%, such as 80%. Thereby, a detected electrical signal corresponding to a maximal detectable electrical signal may indicate that the sample does not contain any analyte. The maximal detectable electrical signal may be determined by applying the portion of reporter reagent without any sample to the measuring compartment.

In an embodiment, the portion of reporter reagent mixed with the sample comprises a surplus of reporter compounds to saturate the capture regions, such as surplus of reporter compounds to saturate the capture regions even when the sample comprises the analyte. Advantageously, the surplus of reporter compounds is up to 50 times of the capture region such as from 2 times to 15 times of the capture region. When applying a surplus of the reporter compounds, the detected electrical signal, may advantageously be detected as a function of time thereby determining the increase of signal over time. It has been found that the rate of the signal increase provides a measure of the amount of reporter compounds which are not bound by analyte and thereby the amount of analyte in the sample can be determined. Further it has been found that the increase of signal over time comprises a linear curve, wherein the slope of the curve may be applied to determine the amount of analyte in the sample. Thereby the amount of analyte in the sample may be determined relatively fast without any need to await a complete reaction between reporter compounds and capture regions.

In the above embodiments, wherein the portion of reporter reagent mixed with the sample comprises a deficient or a surplus of reporter compounds to saturate the capture regions, the surplus or deficient of reporter compounds to saturate the capture regions is advantageously determined as the molar amount of reporter compounds reaching the measuring compartment in relation to the molar amount of capture regions that is immobilized on the electrode. The molar amount of capture regions that are immobilized on the electrode will usually be less than the molar amount of capture reagent or direct capture reagent which at an earlier stage has been supplied to the electrode(s). The molar amount of immobilized capture regions may be determined by using a calibration curve. In the same way also the molar amount of reporter compounds that actually reaches the measuring compartment for a given electrochemical biosensor may be determined. For example if the electrochemical biosensor comprises a filter element or similar a part of the added reporter reagents may be detained in the filter unit and thereby not reaching the measuring compartment.

Both for embodiments of the first arrangement and embodiments of the second arrangement it may be beneficial to dilute the sample where the analyte concentration in the sample is very high, since it may otherwise be difficult to performing an accurate quantitative determination.

As explained above the electrochemical biosensor is a highly versatile electrochemical biosensor, which may be applied in very versatile assays including assays according to the first arrangement or the second arrangement of the method, including methods involving a competing step, a blocking step or a displacement step as well as assays of the method as further described and exemplified below.

The reporter compound may in an embodiment comprise a spacer domain located between the reporter moiety and the capture-binding region. The spacer domain may be relatively short, optionally comprising a backbone comprising up to 20 carbon-atoms e.g. up to 10 carbon-atoms.

The spacer of the reporter compounds may be selected in dependence of the reporter moiety and/or the capture-binding region. In an embodiment, the spacer domain comprises an organic spacer wherein one or more reporter moieties are coupled to sidechains of the organic spacer. In an embodiment, at least two reporter moieties are symmetrically coupled to sidechains of the organic spacer.

The reporter moiety or moieties may comprise a redox mediator, an electrocatalyst, a complexation agent, an enzyme or any combination thereof. Preferably the reporter moiety comprises at least one redox mediator.

methylene blue; methylene blue derivatives; ferrocene; ferrocene derivatives; anthraquinone; anthraquinone derivatives such as anthraquinone-2,6-disulfonate; ruthenium complex, such as ruthenium hexamine and dimethyl sulfoxide ruthenium; ferritin derivative; cobalt; cobalt derivative, such as [Co(GA)2(phen)] (GA=glycolic acid; phen=1,10-phenanthroline); metal nanoparticles, such as nanoparticles comprising at least one of Au, Pt, Pd, Ag or Cu); pyrroloquinoline quinone (PQQ); benzoquine, Osmium(III) complex; diphenylamine; neutral red; toluidine blue; phenosafranine; oxidoreductases or other enzymes capable of electron transfer-reactions or catalysis of redox reactions or any combination comprising at least one of these.

Suitable reporter moiety includes one or more redox mediators selected from methylene blue; methylene blue derivatives; ferrocene; ferrocene derivatives; anthraquinone; anthraquinone derivatives such as anthraquinone-2,6-disulfonate; ruthenium complex, such as ruthenium hexamine and dimethyl sulfoxide ruthenium; ferritin derivative; cobalt; cobalt derivative, such as [Co(GA)2(phen)] (GA=glycolic acid; phen=1,10-phenanthroline); metal nanoparticles, such as nanoparticles comprising at least one of Au, Pt, Pd, Ag or Cu); pyrroloquinoline quinone (PQQ); benzoquine, Osmium(III) complex; diphenylamine; neutral red; toluidine blue; phenosafranine; oxidoreductases or other enzymes capable of electron transfer-reactions or catalysis of redox reactions or any combination comprising at least one of these.

The reporter reagent may comprise additional redox mediator units, wherein the redox mediator units do not comprise the capture-binding region. The additional redox mediator units may conveniently be selected from methylene blue; methylene blue derivatives; ferrocene; ferrocene derivatives; anthraquinone; anthraquinone derivatives such as anthraquinone-2,6-disulfonate; ruthenium complex, such as ruthenium hexamine and dimethyl sulfoxide ruthenium; ferritin derivative; cobalt; cobalt derivative, such as [Co(GA)2(phen)] (GA=glycolic acid; phen=1,10-phenanthroline); metal nanoparticles, such as nanoparticles comprising at least one of Au, Pt, Pd, Ag or Cu); pyrroloquinoline quinone (PQQ); benzoquine, Osmium(III) complex; diphenylamine; neutral red; toluidine blue; phenosafranine; oxidoreductases or other enzymes capable of electron transfer-reactions or catalysis of redox reactions or any combination comprising at least one of these.

Advantageously, the additional redox mediator units differ from the reporter moieties.

The electrical signal(s) may be selected from a potentiometric, amperometric, or impedometric signal. In an embodiment, it may be desired to combine two or more types of electrical signals.

In an embodiment, the at least one electrical signal includes at least a amperometric signal.

The quantitative and/or qualitative detection of the analyte preferably comprises correlating the read-out electrical signal to at least one calibrated value, such as a calibrated threshold and/or calibration curve or function.

Thus, the method may conveniently comprise providing a calibrated threshold and/or calibration curve or function comprising performing an analyte determination on a number X of reference samples with known and different content of the analyte or no analyte at all (blank reference sample). X may conveniently be 2 or more such as at least 5 or more.

Data sets representing for each reference sample the content of analyte and the measured electrical signal or a derivative thereof may advantageously be processed by a mathematical regression analysis to determine a calibration function.

In an embodiment, the method comprises processing the one or more electrical signal, preferably using a computer system as described further below.

Advantageously the method comprises smoothing the at least one electrical signal by subjecting the electric signal to a smoothing processing, preferably the at least one electrical signal comprises at least one of a potentiometric signal, an amperometric signal and/or an impedometric signal and wherein the smoothing processing comprises processing the at least one electrical signal by a smoothing algorithm or such as a moving average, such as simple moving average algorithm (SMA) or a Savitzky-Golay filtering algorithm.

The moving average techniques and algorithm are well known within statistical analyses and have been widely used within trading. The moving average technique is for example describe in “AN ANALYSIS USING SIMULATION TO COMPARE SEVERAL MOVING AVERAGE TECHNIQUES FOR TIME SERIES DATA” by “Hasan et al; Research Square; Jan. 30, 2023; https://www.researchsquare.com/article/rs-2540735/v1 and in Wikipedia, https://en.wikipedia.org/wiki/Moving_average

The moving average algorithm may be a simple moving average algorithm (SMA) or a weighted moving average algorithm (WMA).

Savitzky-Golay filtering algorithm and techniques are for example described in “Methodology and Application of Savitzky-Golay Moving Average Polynomial Smoother” by Ostertagová et al; Global Journal of Pure and Applied Mathematics. ISSN 0973-1768 Volume 12, Number 4 (2016), pp. 3201-3210.

To reduce undesired noise the method advantageously comprises subtracting background noise of the at least one electrical signal to obtain an at least one filtered electrical signal. The method of subtracting background noise, preferably comprises processing the electrical signal(s) comprising fitting the at least one electrical signal to a background correction algorithm, preferably the at least one electrical signal comprises at least one series of electrical signals comprising a plurality of measured data points and wherein the method comprises fitting the electrical signals to the background correction algorithm to obtain a corrected signal.

The background correction algorithm is preferably selected from linear least squares fitting functions such as polynomials; and/or from non-linear least squares fitting functions such as exponentials or power-laws, optional applying initial start guess of at least one parameter, optionally determined directly from the electrical signal.

The series of electrical signals may advantageously be signals as a function of time. In an embodiment, wherein the electrical signals comprises amperometric signals, the current is measured as a function of voltage using square wave voltammetry (SWV) measurement comprising measuring a net current as a function of applied voltage, which may change as a function of time, e.g. 1 Volt/second. In an embodiment, wherein the electrical signals comprises potentiometric signals, the voltage is measured as a function of current, which may be a function of time. In an embodiment, wherein the electrical signals comprises impedometric signals the impedance is measured, which is a complex value comprising a vector sum of a resistive (or real) and a reactive (or imaginary) component. The impedance is for example measured as a function of frequency, which may change as a function of time, thereby providing that the impedometric signals are signals as a function of time.

In an embodiment, the background correction algorithm comprises the formula

y=a*x\hat{ }b+c (1)

When the background correction algorithm comprises a non-linear function, it is desired that the fitting comprising applying at least one start guess, such as a random start guess or a start guess within the order of magnitude of the fitted values of the electrical signals. In an embodiment, the one or more start guesses comprises start guess(es) estimated from the electrical signals, e.g. the raw electrical signals or the electrical signals after being subjected to a smoothing. The one or more start guesses may for example be derived from the determined electrical signals as an example an intersection with the y-axis e.g. where the voltage is zero, for example using data points derived from the first 10% of the electrical signals.

In an embodiment, the at least one filtered signal comprises at least one series of filtered electrical signals comprising a plurality of potentiometric signals, amperometric signals and/or impedometric electrical signals and wherein the method comprises determining a peak height of the filtered signals. The peak height is advantageously determined by
determining a numerically largest value;
performing a linear least squares fitting to one or more functions such as one or more polynomials around a numerically largest values to obtain a local extremum; and/or
performing a non-linear least squares fitting to one or more symmetric and/or asymmetric generalized Gaussian distributions to obtain one or more parametric estimations of the peak height.

In an embodiment, the peak height is determined by performing a fitting to the symmetric Gaussian function:

y=a*exp(−((b−x)/c)\hat{ }2), (2)

wherein a represents the peak height, b and c are fitting parameters and X is the value of the X axis which is advantageously a function of time.

In an embodiment, the at least one filtered signal comprises a plurality of series of electrical signals, wherein each series of electrical signals comprises a plurality of electrical signals selected from potentiometric signals, amperometric signals and/or impedometric signals.

Each of the series of electrical signals may conveniently be determined as consecutive series of electrical signals and wherein the method comprises determining a peak height of each of the series of electrical signals to determine the time dependent peak heights and determining the time-dependent change of peak height by
performing linear least squares fitting to one or more functions such as one or more polynomials; or
performing non-linear least squares fitting to one or more functions such as one or more exponential functions.

The method of processing the signals by determine the time dependent peak heights and determining the time-dependent change of peak height has shown to be very fast and effective.

The plurality of electrical signals selected from potentiometric signals, amperometric signals and/or impedometric signals may advantageously be signals as a function of time.

In an embodiment, the plurality of electrical signals is determined as two or more series of electrical signals. The respective series of electrical signals may advantageously be determined as consecutive series of signals i.e. one after another or the respective series of electrical signals may be partly overlapping, such that some of the electrical signals e.g. up to 50%, such as up to 10% of the electrical signals are included in two series of electrical signals. The series of electrical signals are advantageously filtered by subtracting background noise as described elsewhere herein. Each series of electrical signals may be associated to a time attribute representing the time e.g. t1, t2 etc. or represented by the center time of obtaining the electrical signals, such as the time when half of the signal of a series of electrical signal has been obtained.

It has been found that during the short period of time from obtaining one series of electrical signals to obtaining a next series of electrical signals, the absolute value of the electrical signals increases slightly due to increased hybridization. This results in an increased electrical signal over time which may be determined as a peak height slope. For each consecutive peak height, the peak height slope may be recalculated and this may advantageously be continued until a stable peak height slope is achieved. Thereby a highly accurate determination of the analyte may be obtained.

In an embodiment, the determination of the peak height of each of the series of electrical signals and determining the time-dependent change of peak height comprises determining a peak height slope between a peak height of one series of electrical signals and a next series of electrical signals and recalculating the peak height slope for each of the consecutive series of electrical signals until a stable peak height slope is achieved and deeming the stable peak height slope to be a plateau peak height slope.

In an embodiment, the method comprises estimating a plateau peak height slope by a method comprising processing the peak heights of the respective series of electrical signals comprising
performing a non-linear least squares fitting to one or more functions such as one or more exponential functions or power-law functions;
performing an exponential moving average;
applying Autoregressive Integrated Moving Average; and/or
applying Artificial Intelligence (AI)-assisted predictive machine learning,
   wherein the processing comprises processing peak heights of two consecutive series of electrical signal at a time or processing peak height of three of more series of signals at a time.

Advantageously, the processing is performed for two or more peak heights of series of electrical signals obtained first followed by processing of two or more peak heights of series of electrical signals obtained thereafter, and continuing the process until a stable peak height slope is achieved and deeming the stable peak height slope to be a plateau peak height slope.

The method advantageously comprises determining a concentration of the analyte by correlating the plateau peak height slope to a calibration curve based on measurement on reference samples with known concentration of the analyte. It has been found that the method provide a very accurate determination of the concentration of the analyte in the sample.

In an embodiment, the method also comprises applying a filter element to cover at least the measuring compartment of the electrochemical biosensor or in the alternative the electrochemical biosensor already comprises such filter element as further described below.

The filter element is advantageously applied to cover the measuring compartment prior to supplying the sample to the measuring compartment.

The filter element advantageously comprises a porous filter material, such as a porous filter configured for transfer liquid, such as a sample, a reagent and/or a buffer into the measuring compartment by capillary action.

The porous filter material may thereby be configured for drawing a filtered portion of the sample into the measuring compartment by capillary action. The porous filter material may conveniently filter off undesired particles of the sample, such as particle that otherwise could interfere with the analyte determination in an undesired way.

In an embodiment, the porous filter material is an asymmetrical filter material configured for retention of particles, such as particles larger than a selected size, such a particles which may otherwise influence the detection, such as particles selected from cells (e.g. blood cells), blood clots, aggregates or other particles that may negatively influence the detection.

The filter is in particular desired where the sample comprises relatively large particles, which may potentially influence the detection negatively. By providing the filter to filtering the sample prior to reaching the measuring compartment, a pre-filtering or centrifuging or similar to remove such relatively large particles may be omitted, which simplifies pre-analytical blood sample-handling and ensures that the method becomes less time consuming.

Further the removal of undesired particles from the sample by the filter may reduce experimental and/or electrical noise.

The asymmetrical filter material may preferably have an asymmetrical porosity, such that the filter material comprises relatively small pores located closer to the measuring compartment when applied to cover the measuring compartment and relatively larger pores located at a distance to the measuring compartment, and preferably larger pores located closer to an application surface where the filter is adapted to be contacted with the sample. In an embodiment, the larger pores are located in a section from a surface facing away from the measuring compartment and into a depth of the porous filter material.

In an embodiment, the filter material form part of or constitute a dip stick e.g. a dip stick forming a support as described elsewhere herein, wherein the dip stick comprises a protrusion adapted to dip into the sample for drawing sample into the measuring compartment using capillary action, and wherein the relatively larger pores are located in the dip stick protrusion.

In an embodiment the filter unit comprises reporter compound and wherein the provision of the portion of reporter reagent to the measuring compartment comprises provision of the filter unit with the reporter compound and adding a buffer and/or the sample to the filter unit for resuspending the reporter compound and thereby subjecting of the reporter agent to the measuring compartment.

Due to the porous filter material, not all the added reporter reagent may reach the measuring compartment since some of the reporter reagent or reporter compounds may be detained in the filter material. In an embodiment, the filter material or a part thereof has been wetted with e.g. soaked in reporter reagent and preferably dried prior to providing the filter unit to the electrochemical biosensor. By adding a liquid e.g. the sample or a buffer to the filter material some of the reporter compounds are transported into the measuring compartment. Prior to adding the liquid to the filter material the reporter compounds remain outside the measuring compartment. For further reducing the risk that reporter compounds may enter the measuring compartment and thereby the electrode(s) too soon, the filter material with the dry reporter compounds may be covered by a liquid dissolvable coating, such as a coating that is dissolvable by aqueous liquids, such as the sample and/or the buffer, preferably instantly dissolvable. The coating may e.g. comprises at least one compound selected from vinyl containing compounds, such as polyvinylpyrrolidone, polyvinyl acetate, polyvinyl alcohol and copolymers comprising one or more of the mentioned vinyl compounds; acrylamide/acrylate based polymers or co-polymers thereof, such as octylacrylamide/acrylates/butylaminoethyl methacrylate copolymer and acrylates/T-butylacrylamide copolymer; silicone/siloxane-based polymers such as polydimethylsiloxane; polyethers such as polyethylene glycol; glycerol; ethylene glycol, propylene glycol, and/or any combinations thereof.

The coating may be very thin, such as 0.1 mm or less, such a 0.01 mm or less to ensure that the dissolved coating material does not interfere with the sensing of the electrochemical biosensor. The coating may e.g. be applied using spray coating techniques.

In an embodiment wherein the filter unit comprises reporter compound as dry reporter compound, the filter unit may preferably be obtainable by a method comprising adding a reporter reagent to the porous filter material and drying the porous filter material or by adding the reporter compound as a dry powder optionally encapsulated in a dissolvable pouch.

Thereby the filter unit may form a storage reservoir for the reporter compound. The reporter compound may be stored on or in the porous filter material either at local position or it may be ubiquitous distributed in one or more sections of the porous filter material or in the entire porous filter material.

Storing the reporter compound in or on the filter porous filter material reduces number of consecutive applications of material (“simply add the sample e.g. blood”).

Further, in embodiment where the reporter compound comprises the aptamer, the reaction between reporter compounds and potential analyte in the sample will start already in the porous filter material, which thereby will act as a pre-incubator and ensuring pre-incubation prior to the sample reaches the measuring compartment prior to the sample reaches the measuring compartment.

In embodiments where the capture compound comprises the aptamer the application of the reporter reagent by adding a buffer (e.g. in the form of the sample or another buffer) to the porous filter material may ensure a desired competition between the capture-binding region of the reporter compound and potential analyte in the sample or it may ensure a desired displacement and optional avoid undesired blocking effects.

Advantageously, the filter unit is structured to provide that the porous filter material is located in contact with the working electrode or with a distance up to 5 mm, such as up to 3 mm, such as up to 2 mm determined in dry condition when the filter element is applied to cover the measuring compartment.

By providing that the porous filter material is in direct contact with the working electrode and optionally other electrodes of the measuring compartment or within a short distance up to 5 mm, reduction of diffusion distance may be ensured and thereby a required duration time of an analysis may be reduced, since the closeness of a lower surface of the porous filter material and the one or more electrodes of the measuring compartment may reduce the average diffusion distance of the reporter compound including its reporter moiety to the electrodes compared to a biosensor without a filter, where the reporter compound is available in a droplet placed in or on top of the measuring compartment.

It has been found that the use of the filter element further may provide a desired protection of the measuring compartment and its electrodes from mechanical and airborne damages.

Examples of filters useful for covering the measuring compartment including electrodes of the electrochemical biosensor are for example Cytosep® plasma separation media manufactured by Ahlström, Finland or Vivid™ plasma separation membranes manufactured by Pall/Cytiva, USA.

The method may for example be performed using the electrochemical biosensor described below and/or the electrochemical sensor system described below.

The invention also comprises an electrochemical biosensor for measuring an analyte in a sample. The electrochemical biosensor comprises a measuring compartment for the sample to be analyzed, wherein the measuring compartment comprises a plurality of electrodes, wherein each electrode comprises an electrical lead for providing electrical connections. The electrodes are electrically insulated from each other. The plurality of electrodes comprises at least one working electrode, wherein the working electrode comprises a plurality of immobilized basis compounds, wherein each of the basis compounds comprises an electrode-attachment moiety and a primary region.

The electrical leads are adapted for being applied in electrical contact with an electrochemical reader for read-out of one or more electrical signal.

The measuring compartment and the electrodes may have any shape or configuration, provided that a sample may be provided to the measuring compartment to be in contact with the working electrode and at least one of the other electrodes.

The basis compound may be as described above.

The plurality of electrodes advantageously comprises at least one reference electrode (RE), wherein the reference electrode preferably is located to be in contact with a sample applied in the measuring compartment.

The plurality of electrodes may comprise at least one additional electrode, such as a counter electrode (CE).

In an embodiment, the plurality of electrodes are located to be in contact with a sample e.g. a reference sample or any other liquid applied in the measuring compartment. Optionally the electrochemical biosensor comprises two or more measuring compartments adapted for contacting a same or different liquids.

The liquid may conveniently be an electrolyte solution and/or a sample and/or a reference sample.

For example for providing the at least one calibrated value, such as a calibrated threshold and/or calibration curve or function as described above, measurements may be performed using reference samples comprising known and different concentrations of the analyte, preferably also including a “blank” reference sample without the analyte.

The working electrode may comprise any suitable material, such as the electrode materials known in the art. Examples of preferred electrode materials includes gold, silver, platinum, cobber, titanium, carbon, mercury, tin, or alloys of these, or any combinations comprising one or more of these.

Examples of very suitable electrode materials comprises the following:

Materials for the working electrode: Au, Ag, Pt, Hg, C, modified-C (e.g. metal nanoparticles, Multi- and Single-Walled Carbon Nanotubes, Graphene Oxide etc.), Sn. Materials for the counter electrode: Au, Pt, C, modified-C (e.g. metal nanoparticles, Multi- and Single-Walled Carbon Nanotubes, Graphene Oxide etc.), Sn. Materials for the reference electrode: Au, Ag, Pt, Ag/AgCl, Hg/HgCl, Cu/CuSO4.

For easy and accurate manufacturing, one or more of the electrodes may conveniently be printed electrodes, preferably the electrical leads are printed electrical leads.

The electrodes and/or leads may advantageously be screen printed.

The electrochemical biosensor may in principle have any structural shape or size. For cost reasons it may be advantageously to keep the electrochemical biosensor relatively small, and it has been found that accurate determinations may be obtained even where the required sample volume is very low. Advantageously, the required sample volume is from 1 nL to 100 μL, such as preferably from 20-30 μL.

Examples of structural shapes or sizes of the electrochemical biosensor or electrodes therefor are for example as the gold electrodes market under the name “DropSens by Parque Tecnológico de Asturias-Edif. CEEI. 33428 Lanera (Asturias), Spain or as the shapes or sizes of the electrochemical biosensor marketed by Zimmer and Peacock, Norway.

The electrochemical biosensor may advantageously comprise a support supporting the measuring compartment, wherein the support comprises
a dip stick and the measuring compartment is located at a portion of the dipstick adapted to dip into the sample;
at least one cavity comprising the measuring compartment;
a microfluidic device comprising a microfluidic channel comprising the measuring compartment.

These support structures may provide an easy handling of the electrochemical biosensor.

The support structure may advantageously be adapted to the type of assay to be performed.

The dipstick may advantageously comprise a test strip of a wick material such a cellulose or paper with an end of the strip located in contact with the measuring compartment, such that by dipping the wick material into the sample or dripping sample onto to wick material, a portion of the sample will be transferred into the measuring compartment, such as drawn by capillary action. The wick material is conveniently of an electrically non-conducting material.

The microfluidic device is advantageously a device with a channel with an inlet and a detection site comprising the measuring compartment, the channel is advantageously narrow for supporting a capillary flow.

The cavity may for example be a tube comprising the measuring compartment. In an embodiment, the support comprises a plurality of cavities each comprising a measuring compartment.

The electrochemical biosensor may further comprise antifouling reagents attached to the working electrode and optionally other of the plurality of electrodes, such as all the plurality of electrodes, wherein the antifouling reagents may be as described above.

In an embodiment, the electrochemical biosensor comprises a filter element covering at least the measuring compartment of the electrochemical bio sensor. Preferably the filter element comprises a porous filter material, such as a porous filter configured for transfer liquid, such as a sample, a reagent and/or a buffer into the measuring compartment by capillary action.

The filter element and the porous filter material may advantageously be as described above.

The filter unit may advantageously be arranged such that the porous filter material is located in contact with the working electrode or with a distance up to 5 mm, such as up to 3 mm, such as up to 2 mm determined in dry condition when the filter element is applied to cover the measuring compartment.

The invention also comprises an electrochemical sensor system comprising an electrochemical biosensor as described above and an electrochemical reader.

In an embodiment, the electrochemical biosensor comprises a filter element adapted for covering at least the measuring compartment of the electrochemical biosensor, preferably the filter element comprises a porous filter material, such as a porous filter configured for transfer liquid, such as a sample, a reagent and/or a buffer into the measuring compartment by capillary action.

The filter element may be a separate unit adapted to be mounted to the electrochemical biosensor, e.g. by a click arrangement.

Such click arrangements are well known from mounting one element to another for example the filter unit may comprise a pair of projecting flanges adapted to engage with opposite edges and/or cavities of the electrochemical biosensor to snap into place to a selected position relative to the measuring compartment.

In an embodiment, the filter unit is fixed to and form part of the electrochemical biosensor.

The filter element and/or the porous filter material may be as described above.

The electrochemical reader advantageously comprises an electrical interface adapted for establishing electrical contact with the electrical leads of the respective electrodes of the electrochemical biosensor. The reader is advantageously configured for applying an electrical impact to the electrochemical biosensor and measuring at least one electrical signal selected from a potentiometric, amperometric, or impedometric signal.

The sensor system may further comprise a computer system which preferably comprises a memory storing data representing at least one calibrated value, preferably comprising a calibrated threshold and/or a calibration curve and/or a calibration function.

The calibrated value, calibrated threshold, calibration curve and/or calibration function may be obtained as described above.

The computer system may be a single computer or a group of computers which are in data communication with each other e.g. by wire or wireless. The computer system comprises a processor, such as a multi-core processor.

In an embodiment the computer system or a part thereof is integrated with the electrochemical reader.

The sensor system advantageously, further comprises
a capture reagent and/or
a reporter reagent.

The capture reagent and the capture compounds may be as described above.

The reporter reagent and the reporter compounds may be as described above.

Advantageously, the computer system is configured for obtaining or receiving the at least one electrical signal selected from a potentiometric, an amperometric, an impedometric signal or any combinations comprising one or more of these. The computer system may conveniently be configured for
correlating the measured electrical signal(s) to at least one calibrated value, such as a calibrated threshold and/or calibration curve or function or
processing the measured electrical signal(s) to obtain one or more processed signals and for correlating the processed signal(s) to at least one calibrated value, such as a calibrated threshold and/or calibration curve or function,
   and detecting the analyte of a sample by quantitatively and/or qualitatively determining the analyte in the sample.

The computer system may advantageously be configured for processing the at least one electrical signal by subjecting the electric signal to a smoothing processing, wherein the smoothing processing comprises processing the at least one electrical signal by a smoothing algorithm or such as a moving average, such as simple moving average algorithm (SMA) or a Savitzky-Golay filtering algorithm.

The moving average techniques and algorithm may be as described above.

In an embodiment, the computer system is configured for subtracting background noise of the at least one electrical signal to obtain an at least one filtered electrical signal e.g. as described above.

In an embodiment, wherein the at least one filtered signal comprises at least one series of filtered electrical signals comprising a plurality of potentiometric signals, amperometric signals and/or impedometric electrical signals, the computer system is configured for determining a peak height of the filtered signals e.g. as described above.

In an embodiment, the computer system is configured for determining each of the series of electrical signals as consecutive series of electrical signals and wherein the computer system is configured for determining a peak height of each of the series of electrical signals to determine the time dependent peak heights and determining the time-dependent change of peak height e.g. as described above.

The computer system is advantageously configured for determining a concentration of the analyte by correlating the plateau peak height slope to a calibration curve based on measurement on reference samples with known and concentration of the analyte.

Preferably the reference samples comprises at least two reference samples with different and known concentrations of the analyte, such as at least 3, such as from 5-50 reference samples with different and known concentrations of the analyte.

The invention also comprises a method of producing an electrochemical biosensor for measuring an analyte in a sample, wherein the electrochemical biosensor advantageously is as described above.

The method of producing the electrochemical biosensor comprises
providing a substrate and applying a plurality of electrodes and respective electrical leads to the electrodes to the substrate, wherein the electrodes comprises at least one working electrode (WE), wherein the electrodes are applied to be electrically insulated from each other and to form a measuring compartment adapted for applying a liquid in contact with the electrodes,
cleaning and/or activating at least a surface of the working electrode comprising at least one cleaning cycle of applying a cleaning solution onto the electrode surface, applying a voltage to the working electrode for a selected time slot and washing the electrode using a wash fluid,
providing a basis reagent comprising basis compounds, wherein each of the basis compounds comprise an electrode-attachment moiety and a primary region,
applying the basis reagent to the working electrode and allowing the electrode-attachment moiety of respective basis compound to immobilize to the working electrode.

The electrode may advantageously be printed as described above.

The basis reagent may be as described above.

The measuring compartment is advantageously adapted for applying a liquid, such as a sample or a reference sample in contact with the electrodes as described above. The liquid may conveniently be a reference sample, for example for providing the at least one calibrated value, such as a calibrated threshold and/or calibration curve or function, measurements are performed using fluids comprising different concentrations of the analyte, preferably also including a “blank” liquid without the analyte.

The step of cleaning and/or activating the surface of the working electrode may be time consuming to ensure a very high sensitivity of the electrochemical biosensor. Therefore, it is very beneficial that the universal electrochemical biosensor, which comprises a linker compound as the basis compound as explained above, may be mass produced in a relatively simple way, where after the universal electrochemical biosensor at a later stage may be modified with capture reagent comprising capture compounds directed for a particular analyte and/or reporter compound.

The cleaning has the purpose of removing dirt and dust and the activation has the purpose of ensuring a uniform priming for the following immobilization of basis compounds. The electrode-attachment moiety may advantageously be selected in dependence of the working electrode material and may comprise any suitable moiety capable of chemically immobilize the linker compound to the surface of the working electrode.

In an embodiment, the basis compounds are immobilized using Click chemistry and the electrode-attachment moiety is a moiety forming part of the click attachment. Click chemistry is well known in the art and an example is described in US2019352785.

In an embodiment, the electrode-attachment moiety is as described above.

Advantageously, the electrode-attachment moiety comprises at least one thiol group, such as 2-5 thiol groups, e.g. 3 thiol groups. The attachment moiety may advantageously comprise one or more cysteine amino acids comprising the thiol group(s).

The method may further comprise attaching an antifouling reagents to the working electrode e.g. as described above.

The biosensor production method preferably further comprises producing a filter element and mounting the filter element to cover at least the measuring compartment of the electrochemical biosensor, preferably the filter element comprises a porous filter material, such as a porous filter configured for transfer liquid, such as a sample, a reagent and/or a buffer into the measuring compartment by capillary action.

The filter element and/or the porous filter material is advantageously as described above.

### BRIEF DESCRIPTIONS OF EMBODIMENTS AND EXAMPLES

In the following the invention will be further illustrated by the description of a number of illustrative and non-limiting embodiments and examples of the present invention, with reference to the appended drawings.

The Figs. are schematic and are not drawn to scale and may be simplified for clarity. Throughout, the same reference numerals are used for identical or corresponding parts.

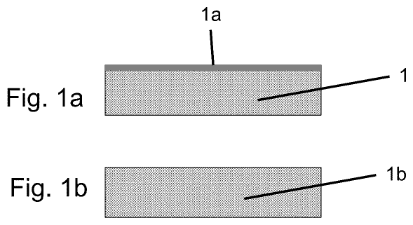

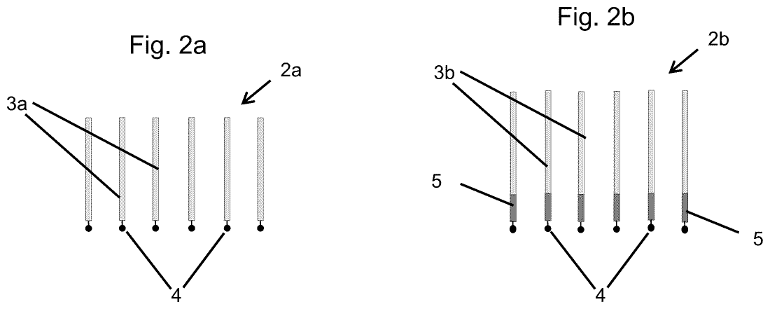

FIG. 1a and FIG. 1b are schematic illustrations of an uncleaned and a cleaned working electrode, which may form part of embodiments of the invention.

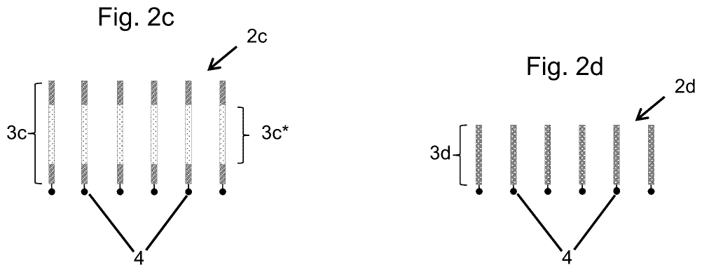

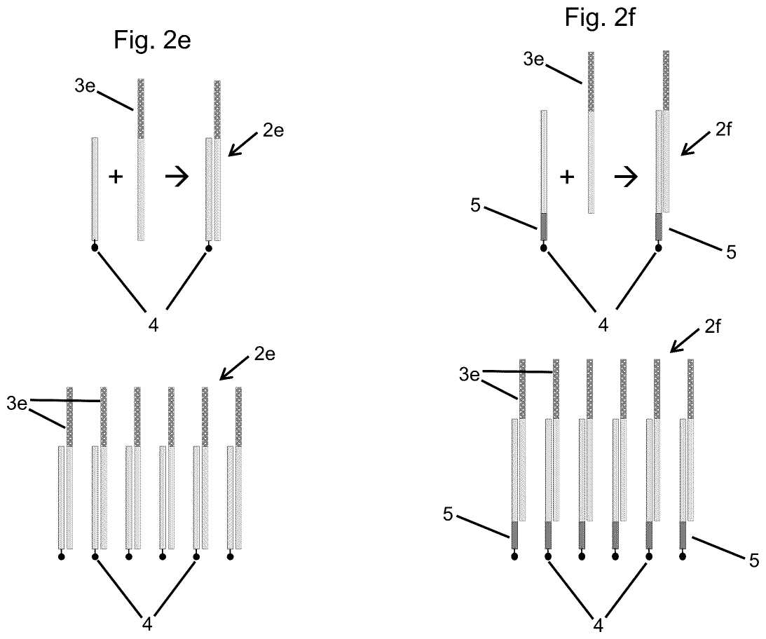

FIG. 2a, FIG. 2b, FIG. 2c, FIG. 2d, FIG. 2e and FIG. 2f are schematic illustrations of basis compounds, which may form part of embodiments of the invention.

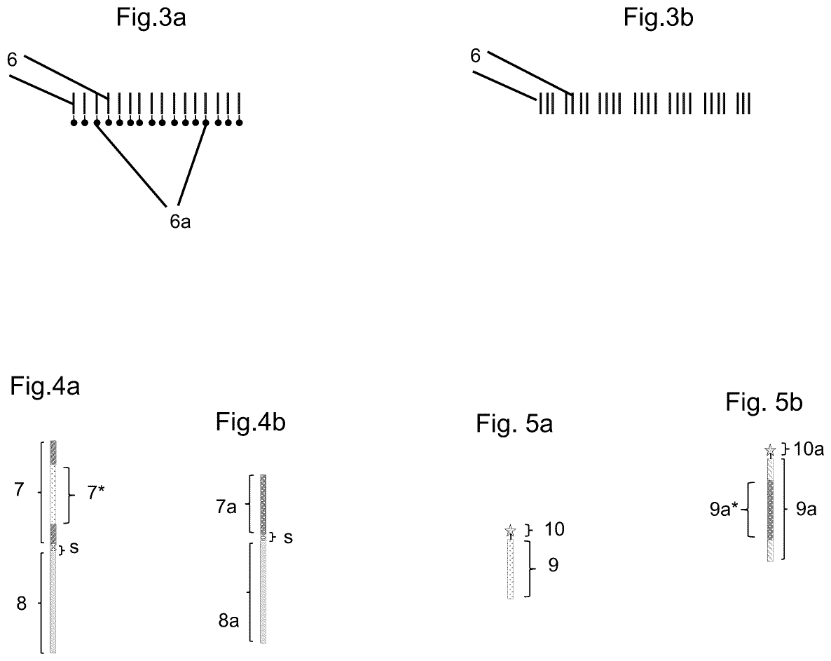

FIG. 3a and FIG. 3b are schematic illustration of antifouling reagents, which may form part of embodiments of the invention.

FIG. 4a and FIG. 4b are schematic illustration of capture compounds, which may form part of embodiments of the invention.

FIG. 5a and FIG. 5b are schematic illustration of reporter compounds, which may form part of embodiments of the invention.

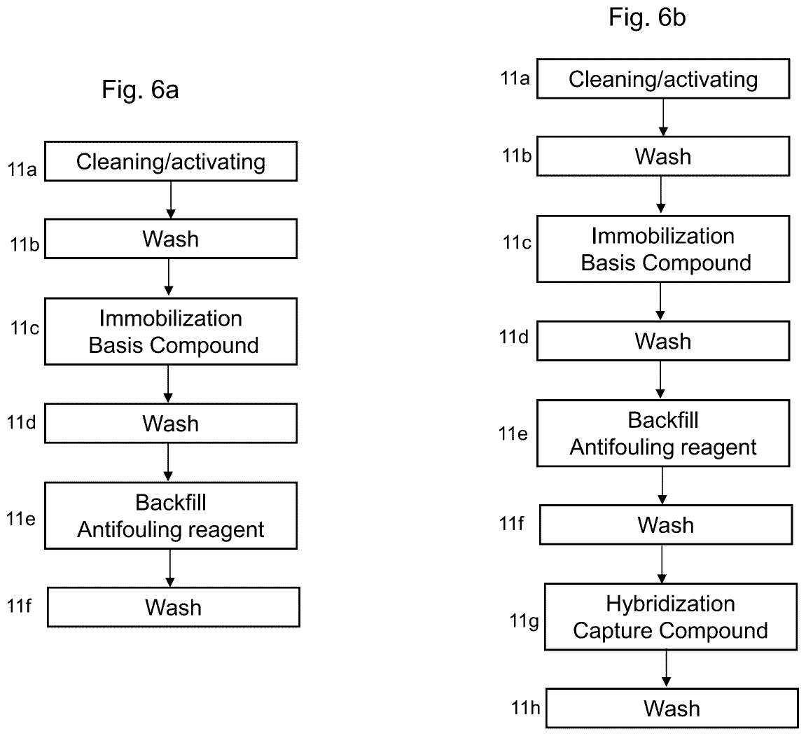

FIG. 6a and FIG. 6b are flow diagrams illustrating steps of embodiments of the procedure for producing electrochemical biosensors of embodiments of the invention with or without capture compounds.

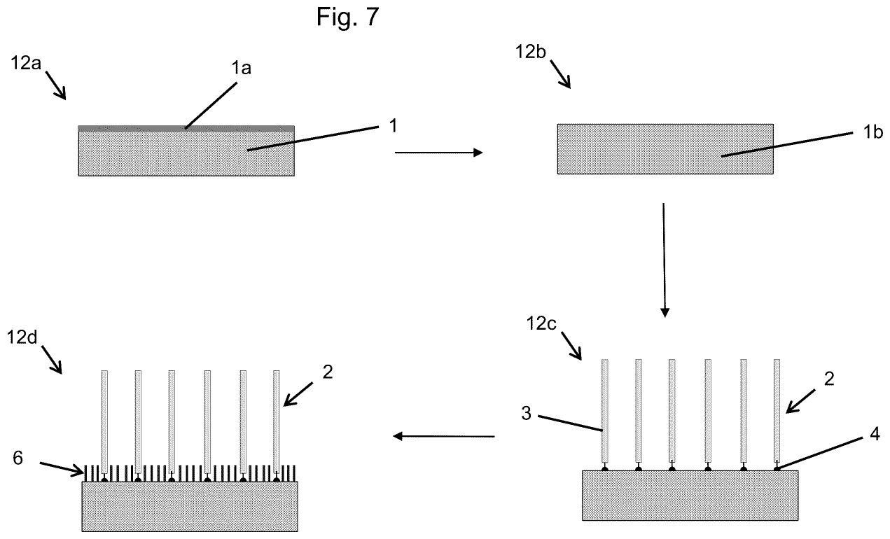

FIG. 7 is a schematic illustration of stages of an embodiment of the procedure for producing an electrochemical biosensor of an embodiment of the invention.

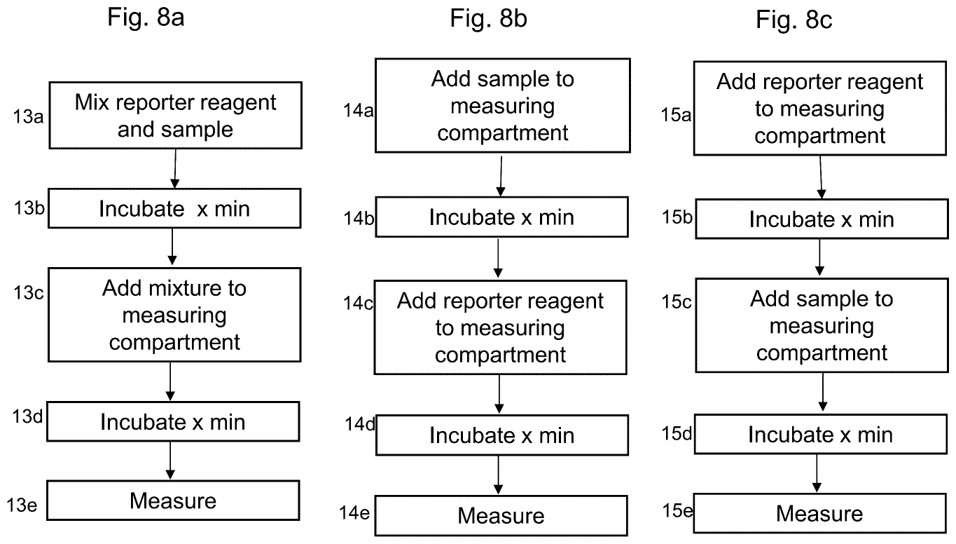

FIG. 8a, FIG. 8b and FIG. 8c are flow diagrams illustrating steps of performing assays of embodiments of the method of the invention in which the capture region of the capture compound comprises the aptamer.

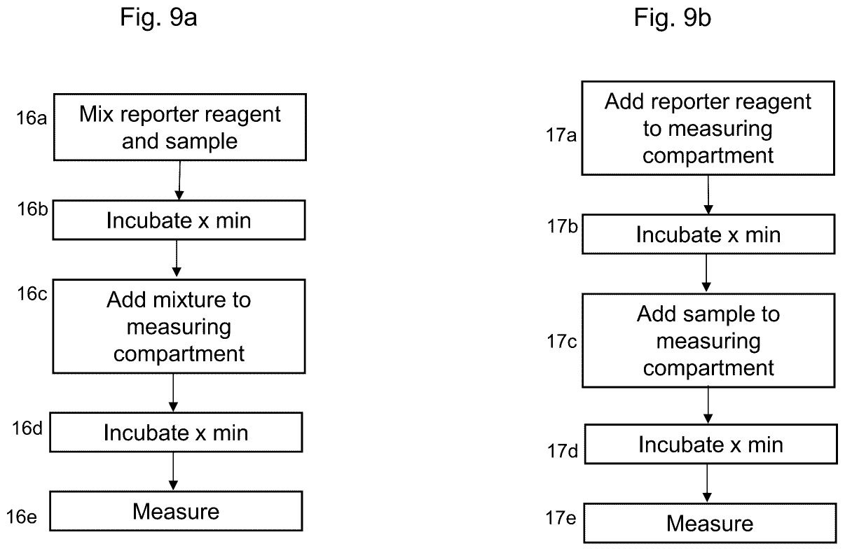

FIG. 9a and FIG. 9b are flow diagrams illustrating steps of performing assays of embodiments of the method of detecting an analyte in a sample of the invention in which the capture-binding region of the reporter compounds comprises the aptamer.

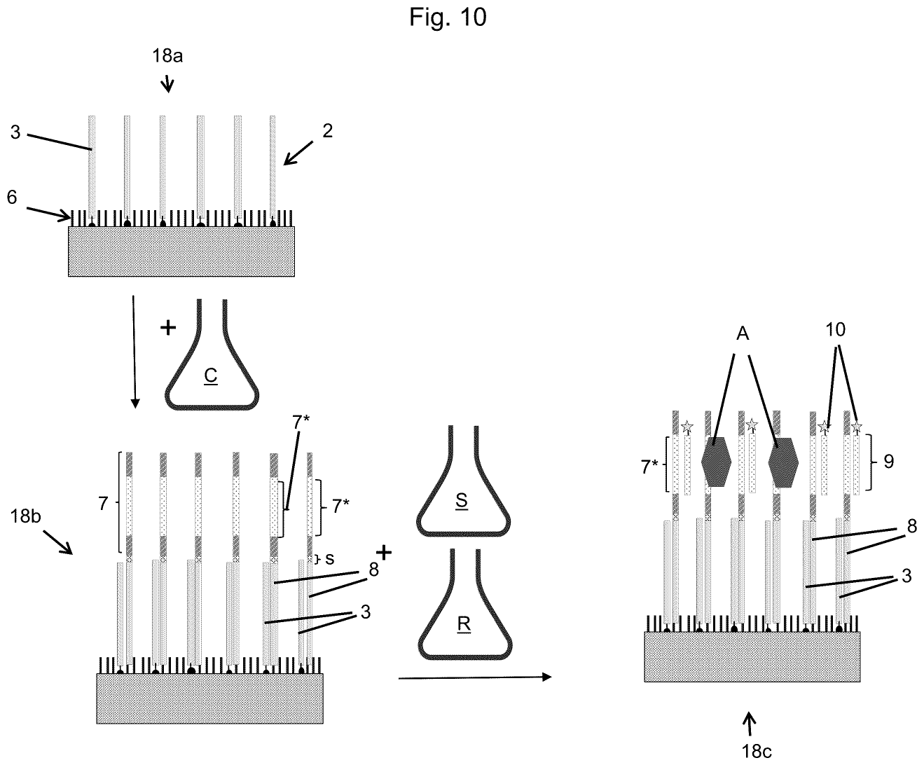

FIG. 10 is a schematic illustration of stages of the procedure for performing an assay of an embodiment of the method of detecting an analyte in a sample of the invention, wherein the capture region of the capture compound comprises the aptamer, and wherein the method comprises a competing step.

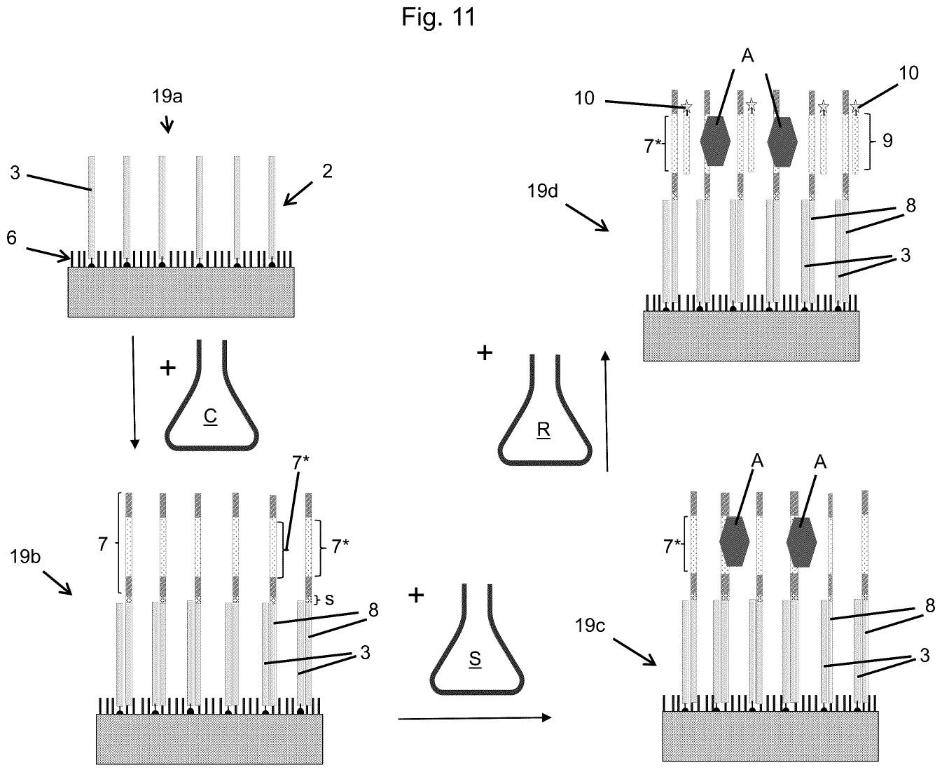

FIG. 11 is a schematic illustration of stages of the procedure for performing an assay of an embodiment of the method of detecting an analyte in a sample of the invention, wherein the capture region of the capture compound comprises the aptamer, and wherein the method comprises a blocking step.

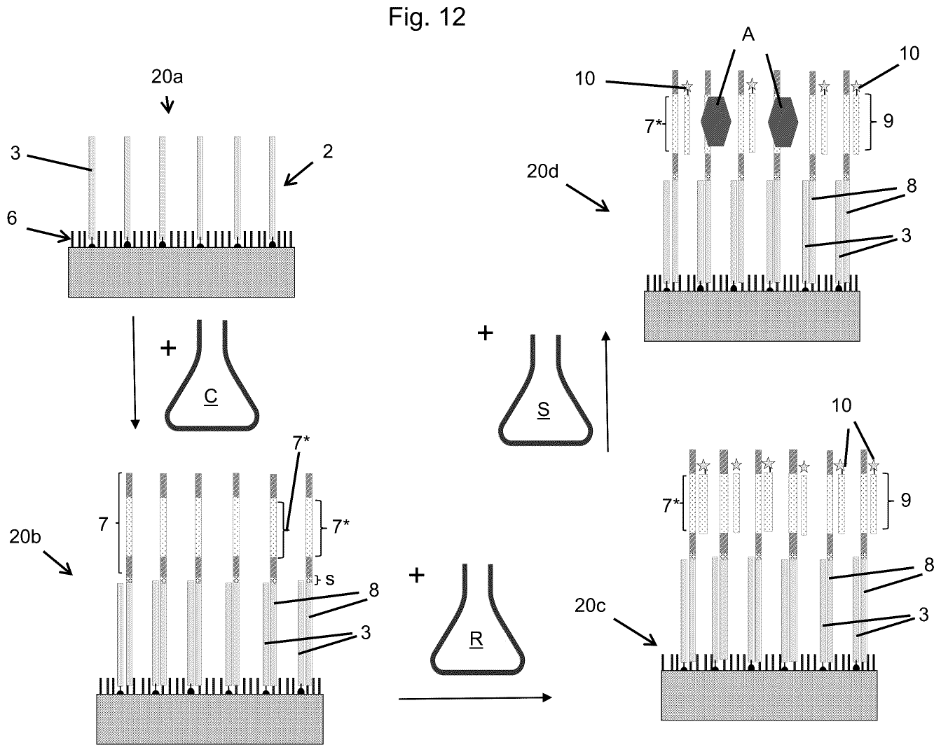

FIG. 12 is a schematic illustration of stages of the procedure for performing an assay of an embodiment of the method of detecting an analyte in a sample of the invention, wherein the capture region of the capture compound comprises the aptamer, and wherein the method comprises a displacement step.

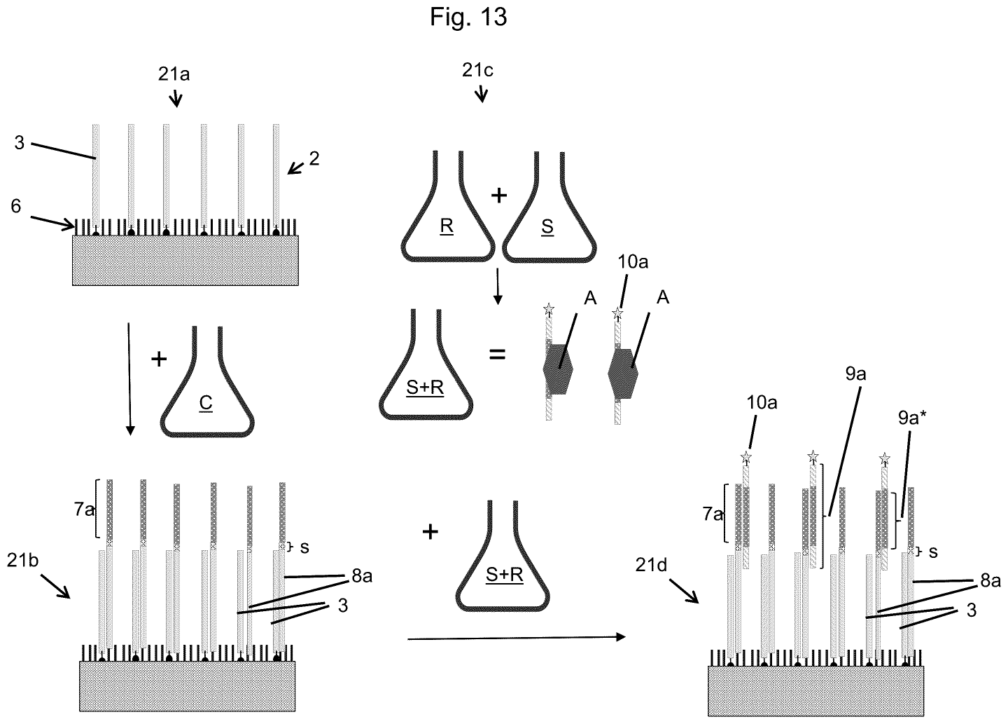

FIG. 13 is a schematic illustration of stages of the procedure for performing an assay of an embodiment of the method of detecting an analyte in a sample of the invention, wherein the capture-binding region of the reporter compounds comprises the aptamer.

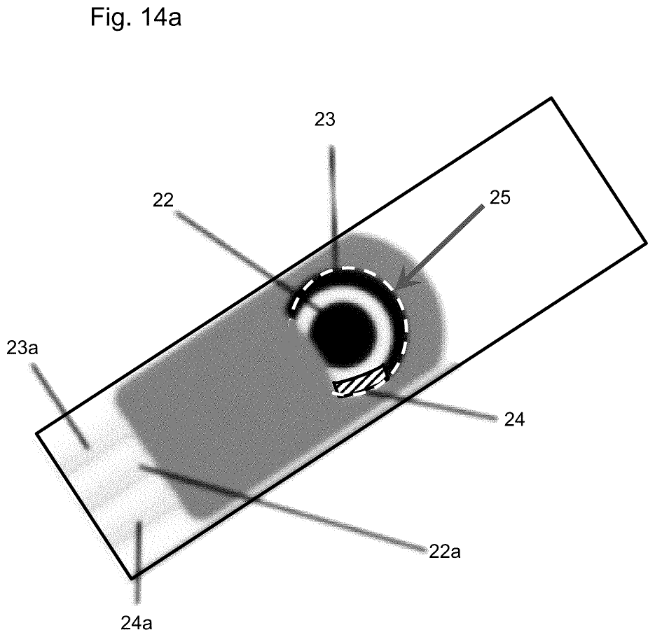

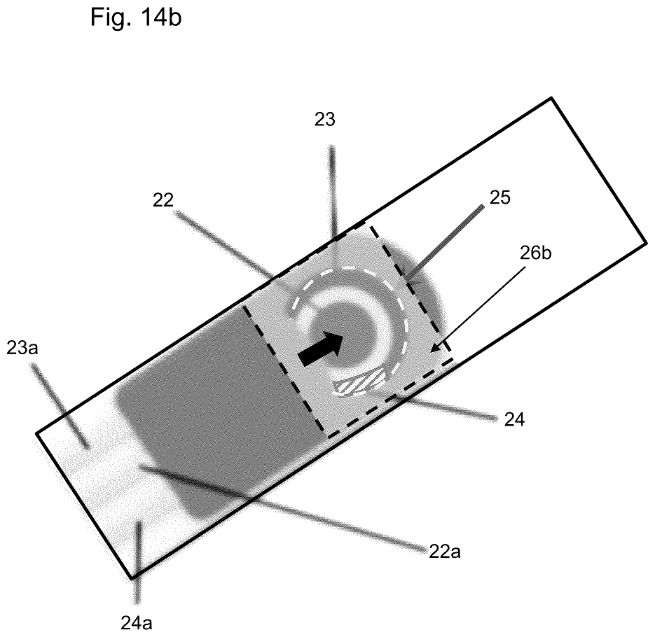

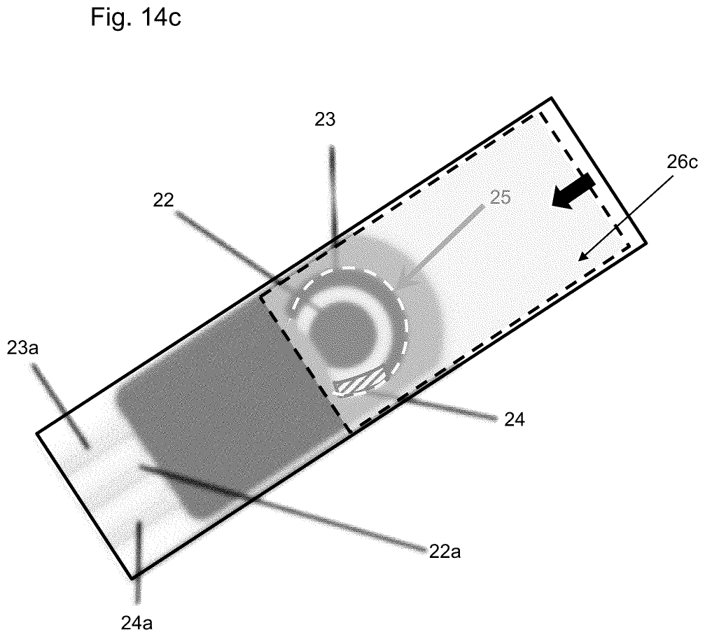

FIG. 14a, FIG. 14b and FIG. 14c illustrate a top view of examples of an electrochemical biosensor of an embodiment of the invention and/or which may form part of embodiments of the invention.

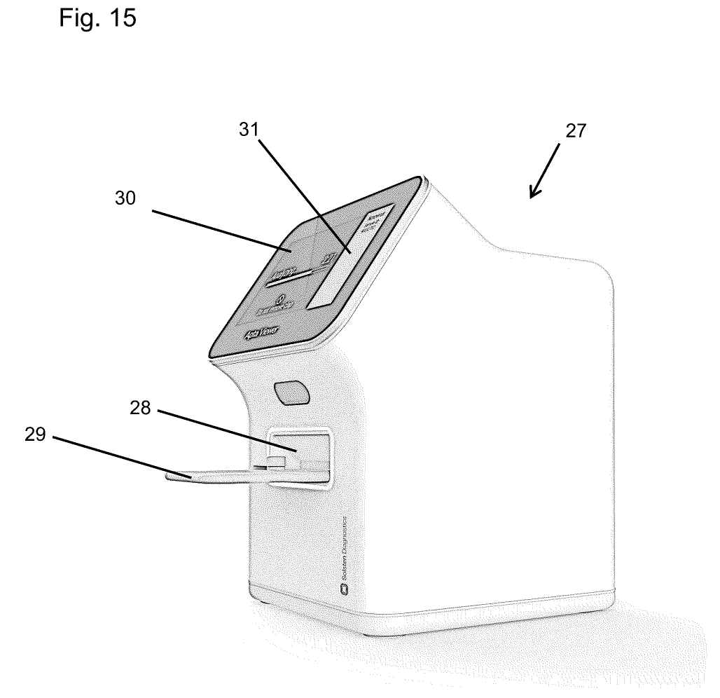

FIG. 15 is a perspective view of an example of an electrochemical reader, which may form part of embodiments of the invention.

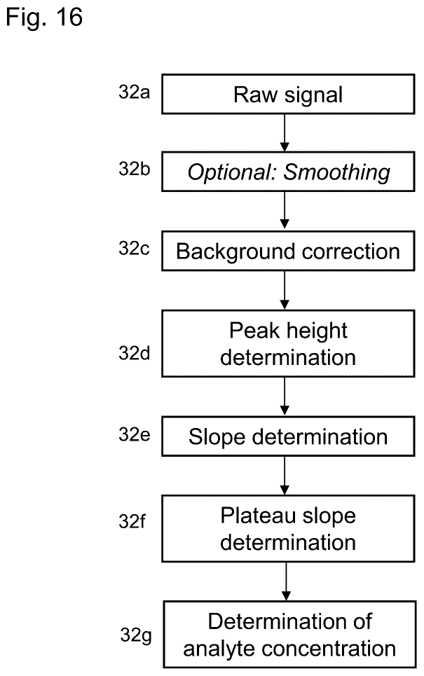

FIG. 16 is a flow diagram illustrating steps of an embodiment of the procedure for processing the potentiometric, amperometric, or impedometric signal to detecting the analyte comprising determining a concentration of the analyte.

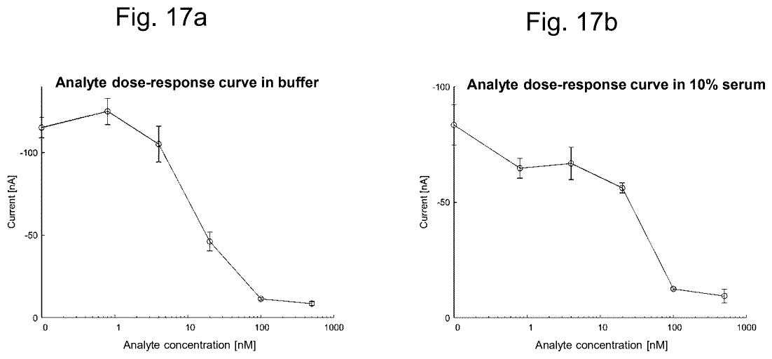

FIG. 17a and FIG. 17b are dose-response curves associated to example 2.

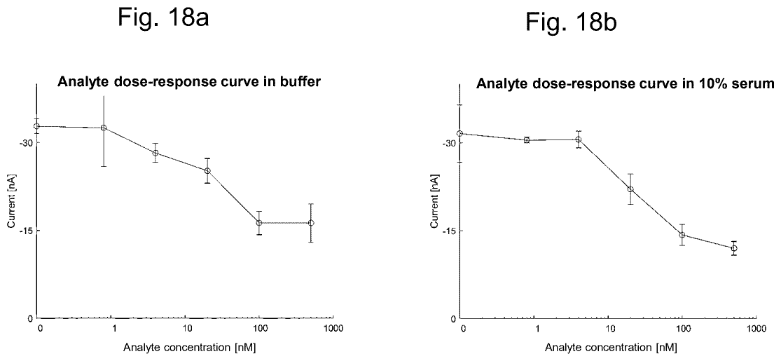

FIG. 18a and FIG. 18b are dose-response curves associated to example 3.

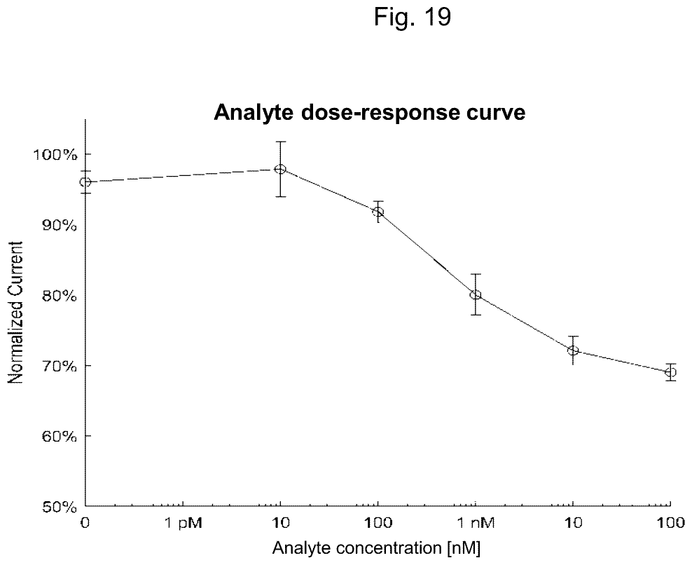

FIG. 19 is a dose-response curve associated to example 4.

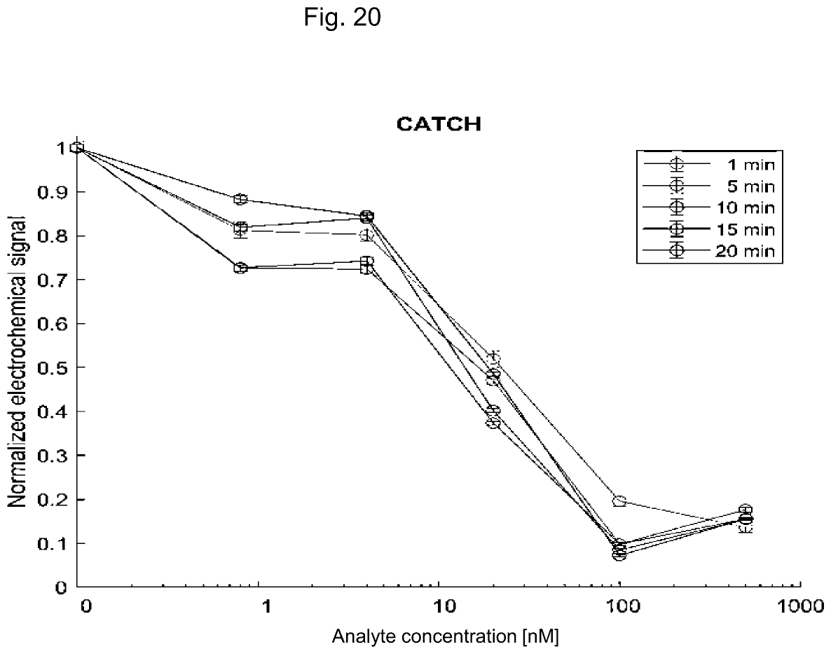

FIG. 20 is a curve of electrochemical signals in dependence of analyte concentration applying different incubation periods associated to example 5.

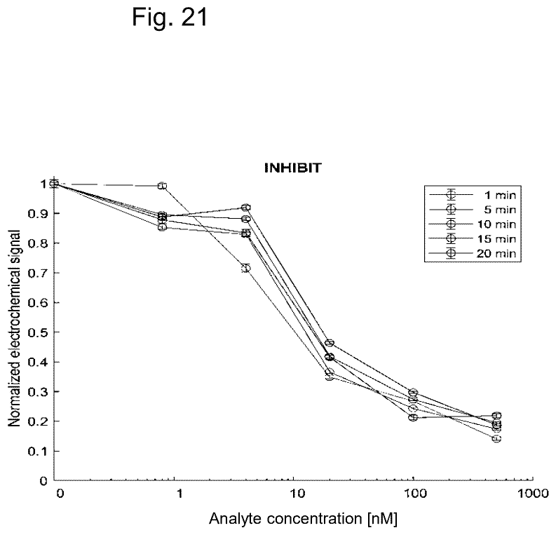

FIG. 21 is a curve of electrochemical signals in dependence of analyte concentration applying different incubation periods associated to example 6.

FIG. 1a illustrates a cross-sectional view of an unclean working electrode 1, which has a dirty or contaminated surface layer 1a. Before immobilizing basis compounds to the surface of the working electrode, the working electrode needs to be cleaned and at the same time the working electrode is activated. In FIG. 1b, the working electrode 1b has been cleaned e.g. by a process as described herein.

FIG. 2a shows a first example of basis compounds, wherein the basis compounds 2a are linker compounds, which each comprises a primary region 3a, which is a linker region and an electrode-attachment moiety 4 adapted for immobilizing the basis compound to a working electrode as described above. The basis compound may comprise a not shown spacer domain as described above. The primary region and the electrode-attachment moiety may be as described above.

FIG. 2b shows a second example of basis compounds, wherein the basis compounds 2b are linker compounds, which each comprises a primary region 3b, which is a linker region, at least one antifouling moiety 5 and an electrode-attachment moiety 4 adapted for immobilizing the basis compound to a working electrode as described above. The basis compound may comprise a not shown spacer domain as described above. The primary region and the electrode-attachment moiety may be as described above.

The at least one antifouling moiety 5 conveniently may be as described above and may provide a first antifouling strategy.

FIG. 2c shows a third example of basis compounds, wherein the basis compounds 2c are direct capture compounds, which each comprises a primary region 3c, which is a capture region and an electrode-attachment moiety 4 adapted for immobilizing the basis compound to a working electrode as described above. In this example, the capture region comprises an aptamer for binding of the analyte and the complementary nucleotides 3c* for hybridization with a capture-binding region of a reporter compound. In this example, the complementary nucleotides 3c* are illustrated as a stretch of nucleotides. However as explained above the conformational shape of the aptamer may provide that the complementary nucleotides 3c*, which thereby may contribute to the hybridizing may or may not be consecutive and for example the nucleotides 3c* of the aptamer which contribute to the hybridizing may be separated by nucleotides that do not contribute to the hybridizing. The basis compound may comprise a not shown spacer domain and/or an antifouling moiety as described above. The primary region and the electrode-attachment moiety may be as described above.

FIG. 2d shows a fourth example of basis compounds, wherein the basis compounds 2d are direct capture compounds, which each comprises a primary region 3d, which is a capture region and an electrode-attachment moiety 4 adapted for immobilizing the basis compound to a working electrode as described above. In this example, the capture region 3d comprises a stretch of nucleotides which may contribute to the hybridizing with a capture-binding region of a reporter compound. The basis compound may comprise a not shown spacer domain and/or an antifouling moiety as described above. The primary region and the electrode-attachment moiety may be as described above.

FIG. 2e and FIG. 2f show a fifth and sixth example of basis compounds, wherein the basis compounds 2e and 2f are pre-hybridized compounds, which each comprises a linker compound comprising a linker region and a capture compound comprising a linker-binding region hybridized with the linker region and a capture region that is the primary region 3e, and an electrode-attachment moiety 4 adapted for immobilizing the basis compound to a working electrode as described above, and where the linker compound of basis compound 2f further comprises at least one antifouling moiety 5. The linker and/or capture compound of the basis compound may comprise a not shown spacer domain as described above. The primary region and the electrode-attachment moiety may be as described above. FIG. 3a illustrates antifouling reagents 6 which each comprises an electrode-attachment moiety 6a adapted for immobilizing the antifouling reagents to the working electrode. Immobilizing the antifouling reagents 6 to the working electrode may provide a second antifouling strategy.

FIG. 3b illustrates antifouling reagents without attachments moieties, which may be applied to the working electrode without being chemically bound and thereby may provide a second antifouling strategy.

The various antifouling strategies may be applied alone or in any combinations.

FIG. 4a shows a first example of a capture compound, wherein the capture compound comprises a linker-binding region 8, a spacer domain s, and a capture region 7. In this example, the capture region comprises an aptamer for binding of the analyte and the complementary nucleotides 7* for hybridization with a capture-binding region. In this example, the complementary nucleotides 7* are illustrated as a stretch of nucleotides. However as explained above the conformational shape of the aptamer may provide that the complementary nucleotides 7* which thereby may contribute to the hybridizing may or may not be consecutive and for example the nucleotides 7* of the aptamer which contribute to the hybridizing may be separated by nucleotides that do not contribute to the hybridizing.

FIG. 4b shows a second example of a capture compound, wherein the capture compound comprises a linker-binding region 8a, a spacer domain s, and a capture region 7a. In this example, the capture region comprises a stretch of nucleotides which may contribute to the hybridizing with a capture-binding region of a reporter compound.

The various regions and/or domains of the capture compounds may be as described above.

FIG. 5a shows a first example of a reporter compound, wherein the reporter compound comprises a capture-binding region 9 and a reporter moiety 10. In this example the capture-binding region 9 comprises a stretch of nucleotides which may contribute to the hybridizing with a capture region of a capture compound.

FIG. 5b shows a second example of a reporter compound, wherein the reporter compound comprises a capture-binding region 9a and a reporter moiety 10a. In this example the capture-binding region 9a comprises an aptamer for binding of the analyte and the complementary nucleotides 9a* for hybridization with a capture region. In this example, the complementary nucleotides 9a* are illustrated as a stretch of nucleotides. However as explained above the conformational shape of the aptamer may provide that the complementary nucleotides 9a* which thereby may contribute to the hybridizing may or may not be consecutive and for example the nucleotides 9a*of the aptamer which contribute to the hybridizing may be separated by nucleotides that do not contribute to the hybridizing.

The various regions, moieties and/or domains of the reporter compounds may be as described above.

FIG. 6a is a flow diagram illustrating steps of the procedure for preparing the electrochemical biosensor with a basis compound, which is a linker compound, a direct capture compound or a pre-hybridized compound.

Referring to FIG. 6a, the procedure of preparing the electrochemical biosensor comprises providing a biosensor structure comprising a measuring compartment comprising a working electrode and optionally additional electrodes as described above. Such biosensor structures are available on market or may be produced as described elsewhere herein.

In step 11a the working electrode is subjected to cleaning. The cleaning may comprise several cycles of applying a cleaning solution onto the electrode surface, applying a voltage to the working electrode for a selected time slot and washing the electrode using a wash fluid e.g. a buffer as illustrated in step 11b. The cleaning may be time consuming and therefore it is very beneficial that the versatile electrochemical biosensor with the linker compound as the immobilized basis compound may be supplied to users or post producers where after the versatile universal electrochemical biosensor at a later stage may be modified with capture reagent comprising capture compounds directed for a particular analyte and/or reporter compound where after the desired assay may be performed without requiring such time consuming cleaning of the working electrode.

In step 11c the basis compound is immobilized to the working electrode, followed by a wash step 11d.

In step 11e antifouling reagents are supplied to the working electrode (backfill), followed by a wash step 11f.

In some instances, steps 11c and 11e may be combined, and thereby also only require one subsequent wash step.

FIG. 6b is a flow diagram illustrating steps of the procedure for preparing the electrochemical biosensor with a basis compound, which is a linker compound and with a capture compound subsequently hybridized with the linker compound.

Referring to FIG. 6b, the procedure of preparing the electrochemical biosensor is as described for FIG. 6a with the additional steps 11g and 11h.

Step 11g comprises providing a capture reagent comprising capture compounds and allowing the linker-binding regions of the capture compounds to hybridize with the primary regions, which are linker regions of the immobilized basis compounds, which are linker compounds. After step 11g, the working electrode of the electrochemical biosensor is washed in step 11h.

FIG. 7 illustrates suitable of stages of the procedure for preparing the electrochemical biosensor.

In stage 12a a dirty working electrode has been provided e.g. as described with reference to FIG. 6a. In stage 12b the working electrode has been cleaned e.g. as also described with reference to FIG. 6a. In stage 12c the basis compounds are immobilized to the working electrode e.g. as described with reference to FIG. 6a. In stage 12d, antifouling reagents has been added to the working electrode e.g. as described with reference to FIG. 6a.

FIG. 8a, FIG. 8b and FIG. 8c are flow diagrams illustrating steps of performing an assay of first arrangement of the method as described above. This first arrangement of the method in which the capture region of the capture compound comprises the aptamer is also referred to as “INHIBIT”.

FIG. 8a illustrates an INHIBIT method comprising a competing step. In step 13a the reporter reagent is mixed with the sample and in step 13b the mixture may be incubated as illustrated (X may be any selected period). Note that potential analytes in the sample will not react with the reporter compounds in the reporter reagent. Thus, this incubation step 13b may be omitted. In step 13c, the mixture is added to the measuring compartment of the electrochemical biosensor and in step 13d it is incubated, e.g. for an incubation period as described above. The capture-binding region of the reporter compounds and optional analyte in the sample will now compete about respectively hybridizing with or binding to the aptamers of the capture region of the capture compounds. After the incubation in step 13d, the electrical signals mal be read out in step 13e. As explained above, the reading out may be initiated before termination of the incubation period, for example the reading out may be initiated immediately after adding the portion of reporter reagent and the sample simultaneously to the measuring compartment.

FIG. 8b illustrates an INHIBIT method comprising a blocking step. In step 14a the sample is added to the measuring compartment and thereafter in step 14b, subjected to an incubation step allowing potential analytes in the sample to be bound by aptamers of the capture region of the capture compounds. Thereby the analytes may block some of the aptamers of the capture regions of the capture compounds from hybridizing with capture-binding regions of reporter compounds of the reporter reagent which is added to the measuring compartment in step 14c. Optionally, the measuring compartment may be washed to remove the sample before adding the reporter reagent. In incubation step 14d, the capture-binding regions of reporter compounds are allowed to react with aptamers of the capture regions of the capture compounds which have not been bound by analytes and thereby been blocked. After the incubation in step 14d, the electrical signals may be read out in step 14e. As explained above the reading out may be initiated before termination of the incubation period, for example the reading out may be initiated immediately after step 14c of adding the reporter reagent to the measuring compartment.

FIG. 8c illustrates an INHIBIT method comprising a displacement step. In step 15a the reporter reagent is added to the measuring compartment and thereafter in step 15b, subjected to an incubation step allowing capture-binding regions of reporter compounds of the reporter reagent to hybridize with aptamers of the capture regions of the capture compounds. In step 15c, the sample is added to the measuring compartment and subjected to an incubation period in step 15d. Optionally, the measuring compartment may be washed to remove the excess reporter reagent before adding the sample. Potential analytes of the sample will now be allowed to bind to aptamers of non-hybridized capture regions of the capture compound including de-hybridized capture regions in which a hybridized capture-binding region has been displaced.

For an optimal electrical read out, it may be desired that most of the aptamers of the capture region are hybridizing with capture-binding regions of reporter compounds in step 15a/15b. In steps 15c/15d the analytes may then—due to higher binding affinity to the aptamers—displace a relative large number of reporter compounds.

After the incubation in step 15d, the electrical signals mal be read out in step 15e. As explained above, the reading out may be initiated before termination of the incubation period, for example the reading out may be initiated immediately after step 15c of adding the sample to the measuring compartment.

FIG. 9 and FIG. 9b are flow diagrams illustrating steps of performing an assay of second arrangement of the method as described above. This second arrangement of the method in which the capture-binding region of the reporter compounds comprises the aptamer is also referred to as “CATCH”.

FIG. 9a illustrates a CATCH method comprising a reporter compound blocking step. In step 16a the reporter reagent is mixed with the sample and in step 16b the mixture may be incubated as illustrated. The aptamers of the capture-binding regions of the reporter compounds will now be allowed to bind to potential analytes in the sample. In step 16c, the mixture is added to the measuring compartment and allowed an incubation period in step 16d. Aptamers of the capture-binding regions of the reporter compounds that have not bound to analytes will now be available for hybridization with capture regions of the capture compounds. The amount of reporter compounds in the portion of reporter reagent mixed with the sample, is advantageously selected such, that it can be detected if even a small part of the reporter compounds has been blocked by analytes in steps 16a/16b.

Since the analytes usually are much larger than the reporter compounds, the mixture of the sample and the reporter reagent may conveniently be filtered to remove analytes and analytes bound to reporter compounds before adding the mixture to the measuring compartment.

After the incubation in step 16d, the electrical signals mal be read out in step 16e. As explained above, the reading out may be initiated before termination of the incubation period, for example the reading out may be initiated immediately after step 16c of adding the mixture to the measuring compartment.

FIG. 9b illustrates a CATCH method comprising a displacement step. In step 17a the reporter reagent is added to the measuring compartment and thereafter in step 17b, subjected to an incubation step allowing aptamers of capture-binding regions of reporter compounds of the reporter reagent to hybridize with capture regions of the capture compounds. In step 17c, the sample is added to the measuring compartment and subjected to an incubation period in step 17d. Potential analytes of the sample will now displace the hybridization between capture regions of the capture compounds and aptamers of capture-binding regions of reporter compounds and will bind to the displaced aptamers of capture-binding regions of reporter compounds.

To optimize the electrical signal, it may be desired to remove non-hybridized reporter compounds from the measuring compartment e.g. by washing with a buffer, prior to adding the sample in step 17c.

After the incubation in step 17d, the electrical signals may be read out in step 17e. As explained above, the reading out may be initiated before termination of the incubation period, for example the reading out may be initiated immediately after step 17c of adding the sample to the measuring compartment.

FIG. 10 illustrates suitable stages of an INHIBIT method, wherein the method comprises a competing step. In stage 18a capture reagent C and an electrochemical biosensor with a working electrode 1a comprising immobilized linker compounds 2 as basis compounds and antifouling reagents have been provided. In step 18b the capture reagent C has been added to the measuring compartment of the electrochemical biosensor comprising the working electrode 1a, and the linker-binding regions 8 of the capture compounds have hybridized with the primary regions, which are linker regions 3 of the basis compounds, which are linker compounds. Between stage 18b and stage 18c a sample S and a reporter reagent R have been provided and added simultaneously to the measuring compartment of the electrochemical biosensor comprising the working electrode 1a. As explained above the sample S and the reporter reagent R may be mixed prior to being added to the measuring compartment.

In step 18c, the capture-binding region 9 of the reporter compounds and analytes A of the sample have competed about respectively hybridizing with and binding to the aptamers of the capture regions 7 of the capture compounds and it can be seen that a first portion P1 of aptamers of the capture regions 7 have hybridized with capture-binding regions 9 of reporter compounds and a second portion P2 of aptamers of the capture regions 7 has bound to analytes A. The portions P1 and P2 depends on the concentration of analytes A in the sample and by reading out the electrical signals the concentration of analytes A in the sample may be determined for example by correlating the read electrical signals or derivative thereof with a calibration curve or function.

FIG. 11 illustrates suitable stages of an INHIBIT method, wherein the method comprises a blocking step. The first two stages 19a, 19b correspond to or are identical with the first two stages 18a, 18b of the INHIBIT method comprising a competing step shown in FIG. 10.

Between stage 19b and stage 19c a sample S has been provided and added to the measuring compartment of the electrochemical biosensor comprising the working electrode 1a. In step 19c, analytes A of the sample have been bound by a portion P1 of the aptamers of the capture regions 7 of the capture compounds.

Between stage 19c and stage 19d, a reporter reagent R has been provided and added to the measuring compartment of the electrochemical biosensor comprising the working electrode 1a. In step 19c, capture-binding regions 9 of reporter compounds of the reporter reagent R have hybridized with a portion P2 of the aptamers of the capture regions 7 of the capture compounds. Before adding the reporter reagent R, the analytes A of the sample had been bound by a portion P1 of the aptamers of the capture regions 7 of the capture compounds and thereby blocked these aptamers from hybridizing with the capture-binding regions 9 of the reporter compounds, and thereby reducing the number of aptamers of capture regions 7 available for hybridization with the capture-binding regions 9 of the reporter compounds. The portions P1 and P2 thereby depends on the concentration of analytes A in the sample and by reading out the electrical signals the concentration of analytes A in the sample may be determined for example by correlating the read electrical signals or derivative thereof with a calibration curve or function.

FIG. 12 illustrates suitable stages of an INHIBIT method, wherein the method comprises a displacement step. The first two stages 20a, 20b correspond to or are identical with the first two stages 18a, 18b of the INHIBIT method comprising a competing step shown in FIG. 10.

Between stage 20b and stage 20c a reporter reagent R has been provided and added to the measuring compartment of the electrochemical biosensor comprising the working electrode 1a. In step 20c, capture-binding regions 9 of reporter compounds of the reporter reagent R have hybridized with a portion larger than P2 of the aptamers of the capture regions 7 of the capture compounds. In the shown embodiment, capture-binding regions 9 of reporter compounds of the reporter reagent R have hybridized with most of or even all of the aptamers of the capture regions 7 of the capture compounds.

Between stage 20c and stage 20d, a sample S has been provided and added to the measuring compartment of the electrochemical biosensor comprising the working electrode 1a. In step 20d, analytes A of the sample have displaced some of the reporter compounds which have de-hybridized allowing the analytes A to be bound by a portion P1 of the aptamers of the capture regions 7 of the capture compounds and reducing the capture-binding regions 9 of reporter compounds of the reporter reagent R to be hybridized with a portion P2 of the aptamers of the capture regions 7 of the capture compounds. The portions P1 and P2 thereby depends on the concentration of analytes A in the sample and by reading out the electrical signals the concentration of analytes A in the sample may be determined for example by correlating the read electrical signals or derivative thereof with a calibration curve or function.

FIG. 13 illustrates suitable of stages of a CATCH method, wherein the method comprises a reporter compound blocking step.

In stage 21a, capture reagent C and an electrochemical biosensor with a working electrode 1a comprising immobilized basis compounds, which are linker compounds 2 and antifouling reagents have been provided. In step 21b the capture reagent C has been added to the measuring compartment of the electrochemical biosensor comprising the working electrode 1a, and the linker-binding regions 8a of the capture compounds have hybridized with the primary regions, which are linker regions 3 of the basis compounds, which are linker compounds.

In stage 21c, a reporter reagent R and a sample S have been provided and mixed with each other. Thereby analyte A of the sample have been allowed to be bound by aptamers of the capture-binding regions 9a of the reporter compounds of the reporter reagent R. Thereby the reporter compounds in which the capture-binding region 9a have been bound by analytes A will be blocked from hybridizing with the capture regions 7a of the capture compound when added to the measuring compartment.

In stage 21d, the mixed reporter reagent R and sample S have been added to the measuring compartment and the non-blocked capture-binding regions 9a of the reporter compounds have been allowed to hybridize with the capture regions 7a of the capture compound. As mentioned above the mixture of reporter reagent R and sample S may be filtered prior to adding to the measuring compartment.

The higher concentration of analyte A in the sample, the more of the reporter compounds will be blocked by the capture-binding region 9a being bound to analytes A. By reading out the electrical signals the concentration of analytes A in the sample may be determined for example by correlating the read electrical signals or derivative thereof with a calibration curve or function.

The electrochemical biosensor shown in FIG. 14a comprises a working electrode 22, a counter electrode 23 and a reference electrode 24. The working electrode 22, the counter electrode 23 and the reference electrode 24 are located in a measuring compartment 25 illustrated by a dotted line. The electrochemical biosensor further comprises a first electrical lead 22a for the working electrode 22, a second electrical lead 23a for the counter electrode 23 and a third electrical lead 24a for the reference electrode 24. The electrical leads 22a, 23a, 24a are adapted for providing electrical contact with an electrochemical reader for reading out electrical signal of the electrochemical biosensor.

The electrochemical biosensor shown in FIG. 14b is as the electrochemical biosensor shown in FIG. 14a, but where a filter unit 26b has been mounted on top of the measuring compartment 25. The sample is applied on top of the filter unit over the measuring compartment comprising the working electrode as indicated by the short fat arrow.

The electrochemical biosensors shown in FIG. 14c is as the electrochemical biosensor shown in FIG. 14a, but where a filter unit 26c has been mounted on approximately half of the biosensor surface including the measuring compartment 25 and distant to the electrical leads 22a, 23a, 24a. The sample is applied on top of the filter unit distant from the working electrode as indicated by the short fat arrow.

The filter unit may be as described above.

The electrochemical reader 27 shown in FIG. 15 comprises a slot 28 for an electrochemical biosensor. In the shown example an electrochemical biosensor 29 has temporarily been inserted in the slot 28. The electrochemical reader 27 further comprises an operating panel 30 and a display 31. The electrochemical reader further comprises a not shown computer which conveniently form part of the computer system of the electrochemical sensor system.

FIG. 16 is a flow diagram illustrating steps of a preferred procedure for processing the potentiometric, amperometric, or impedometric signal to determine the concentration of the analyte in the sample with a desired high accuracy and wherein the determination may be performed relatively fast.

In step 32a one or more potentiometric, amperometric, and/or impedometric signals (in the FIG. 16 referred to as “raw signal”) are collected from the biosensor as described above for being processed by the computer system e.g. by processing the signals using sequential algorithms.

The raw signal may advantageously comprise at least one series of electrical signals.

In step 32b the one or more collected signal are processed by a smoothing algorithm such as simple moving average method (SMA) or Savitzky-Golay filtering. This step 32b is an optional step.

In step 32c the collected, optionally smoothened signals are corrected for background by a process comprising subtracting background noise e.g. as described. The background correction may advantageously comprise filtering the signals from background noise determined by linear least squares fitting of one or more functions such as polynomials; or non-linear least squares fitting of one or more functions such as exponentials or power-laws with initial parameters determined directly from the electric signal.

In step 32d the peak height of the collected, optionally smoothened, background-corrected signal determined for example by a process comprising determining the numerically largest value; or by applying linear least squares fitting of one or more functions such as polynomials around the numerically largest values to obtain local extremum; or by performing non-linear least squares fitting of one or more symmetric and/or asymmetric Gaussian function or Gaussian-derivative functions preferably comprising a peak shapes to obtain parametric estimations of the peak height.

In step 32e the peak height of the most recent collected, optionally smoothened, background-corrected signals are analyzed together with the peak heights of optionally smoothened, background-corrected signals collected previously from the same biosensor in order to determine the slope for the time-dependent change of peak height by linear least squares fitting of functions such as polynomials; or non-linear least squares fitting of functions such as exponentials.

In step 32f the plateau slope is estimated as described above e.g. by forecasting using non-linear least squares fitting of functions such as exponentials or power-laws; or exponential moving average; or Autoregressive Integrated Moving Average; or artificial intelligence (AI)-assisted predictive machine learning.

In step 32g the estimated plateau slope is used to determine the analyte concentration calculated from an internally stored calibration curve for example as described above.

### EXAMPLES

In the following is provided a number of illustrative and non-limiting examples of the invention

### General about Aptamers and Oligonucleotide Containing Compounds

All oligonucleotides including aptamers were manufactured by IDT, Belgium/US or LGC Biosearch, Denmark. All applied oligonucleotides were purified by HPLC by the supplier. When thiol modified oligonucleotides were purchased, they were delivered as disulfides. The disulfide was reduced to the free thiol by tris (2-carboxyethyl)phosphine (TCEP).

### Example 1—Biosensor Production on Screen Printed Gold Electrodes

A universal electrochemical biosensor comprising a measuring compartment was produced from a biosensor substrate comprising screen-printed gold electrodes by the following steps.

### Cleaning and Activation

The screen-printed gold electrodes (SPE) were washed in isopropanol for 1 minute and washed with water. The SPE was connected to a potentiostat and immersed in a cleaning and activation solution containing 0.15 M H₂SO₄. Potential sweeping was performed from −0.2 V to 1.2 V for electrochemical conditioning of the gold surface.

The cleaning and activation protocol is essential for obtaining a functional biosensor.

### Immobilization of Basis Compound

The basis compound with a free thiol as attachment moiety was dissolved in a buffer consisting of DPBS+MgCl₂, pH=7.4. This basis reagent was added to the electrodes and incubated at room temperature in a humid chamber. The electrodes were washed with the buffer prior to the next step.

### Antifouling

The chosen antifouling reagent, preferably containing a free thiol, such as mercaptohexanol, was dissolved in a buffer consisting of DPBS+5 mM MgCl₂, pH=7.4, to a concentration of for example 1 mM. This mixture was added to the electrode and incubated at room temperature for 120 minutes in a humid chamber. The electrodes were washed with the buffer prior to the next step.

After this step the electrodes can be dried and stored dry and cold if they are not used immediately.

### Example 2—Biosensor Assay-Detection of Analyte (CATCH)

The biosensor was applied for the detection of an analyte, such as thrombin.

The biosensor was produced with the immobilized basis compound being a linker compound 2a as shown in FIG. 2a and 1 mM mercaptohexanol (MCH) as antifouling reagent. It was converted to an analyte specific biosensor by addition of a capture compound as shown in FIG. 4b. The capture compound comprises a linker-binding region, which is complementary to the linker region of the immobilized linker compound and the capture region which is complementary to a part of the aptamer of the reporter compound of the reporter reagent. The capture compound was dissolved in a buffer (DPBS+5 mM MgCl₂, pH=7.4) and this capture reagent was added to the universal biosensor electrodes. After incubation for 60 minutes the capture compound was bound to the linker region through hybridization.

In separate vials different concentrations of analyte were mixed with a fixed concentration of reporter compound (50 nM) of the type shown in FIG. 5b comprising an aptamer 9a and incubated for 5 minutes. In the present example, the aptamer of the reporter compound was specific for the analyte, thrombin. Thereafter, this mixture was added to the biosensor electrodes. Each concentration of analyte was analyzed in triplicate on separate biosensors. After 10 min incubation the electrochemical signal was measured by square wave voltammetry.

In this assay the reporter compound contains the analyte-specific aptamer. Thereby, an increasing amount of analyte will result in decreasing current due to a reduction of free reporter compounds in the solution which can hybridize with the capture region of the capture compound.

The results are shown as a dose-response curve in FIG. 17a. The highest electrochemical signal is obtained for analyte-free solutions. The presence of analyte results in a characteristic S-shaped curve with decreasing electrochemical signal at increasing analyte concentrations. A complete quenching of the electrochemical signal was found around 100 nM of analyte.

As shown in FIG. 17b, this assay was also demonstrated to work in 10% serum with a ˜40% decrease in signal and some loss of sensitivity. It is expected that using additional antifouling strategies, such as the described first and/or second antifouling strategies will increase the sensitivity.

### Example 3—Biosensor Assay-Detection of Analyte (INHIBIT/Blocking)

The biosensor was applied for the detection of an analyte, such as thrombin.

The biosensor was produced with the immobilized basis compound being a linker compound 2a as shown in FIG. 2a and 1 mM mercaptohexanol (MCH) as antifouling reagent and was converted to an analyte specific biosensor by addition of the capture compound as shown in FIG. 4a, which comprises the aptamer and a linker-binding region which is complementary to the linker region of the immobilized linker compound. In the present example, the aptamer of the capture compound was specific for the analyte, thrombin. The capture compound was dissolved in a buffer (DPBS+5 mM MgCl₂, pH=7.4) and this capture reagent was added to the universal biosensor electrodes. After incubation for 60 minutes the capture compound was bound to the linker region through hybridization.

In this assay the reporter compound is complementary to a part of the capture region which comprises the analyte-specific aptamer.

Different concentrations of analyte were incubated for 5 minutes on a biosensor. Each thrombin concentration was analyzed in triplicate on separate biosensors. Thereafter, a fixed concentration of reporter compound (100 nM) was added to the biosensor. After 10 min incubation the current was measured by square wave voltammetry.

In this assay the hybridization of the reporter oligonucleotide to the capture region will be blocked by the analyte, which is bound to the aptamer of the capture region. An increasing amount of analyte will result in decreasing current due to fewer bound reporter compounds.

The results are shown as a dose-response curve in FIG. 18a. The highest electrochemical signal is obtained for analyte-free solutions. The presence of analyte results in a characteristic S-shaped curve with decreasing electrochemical signal at increasing analyte concentrations until a background electrochemical signal is detected.

As shown in FIG. 18b, this assay was also demonstrated to work in 10% serum.

### Example 4—Biosensor Assay-Detection of Analyte (INHIBIT/Displacement)

The biosensor was applied for the detection of an analyte, such as thrombin.

The biosensor was produced with same immobilized basis compound and antifouling reagents and was converted to an analyte specific biosensor as in example 3.

In this assay, a fixed concentration of reporter compound (100 nM) was added to the biosensor. The hybridization of the reporter compound with the capture compound was followed by consecutive measurements of square wave voltammetry. After sufficient time (˜20 min) a fixed concentration of analyte was added to the existing volume of reporter compound. Each thrombin concentration was analyzed in triplicate on separate biosensors.

In this assay the capture-binding region of the reporter compound will hybridize with the capture region of the capture compound. The added target will result in a displacement of the reporter compound. An increasing amount of target will result in decreasing current due to fewer bound reporter compounds. The relative decrease of steady state current after addition of target is determined and is related to the amount of displaced reporter compound.

The results are shown as a dose-response curve in FIG. 19. The highest electrochemical signal is obtained for analyte-free solutions. The presence of analyte results in a characteristic decrease in the electrochemical signal until a background electrochemical signal is detected.

### Example 5—Biosensor Assay-Incubation Time of Reporter Compound (CATCH)

The biosensor was produced with the same immobilized basis compound and antifouling reagents and was converted to a thrombin specific biosensor as in example 2.

Here the incubation time of the mix of analyte and reporter compound was varied from 1 minute to 20 minutes.

The assay was demonstrated to work equally well after only 1 min incubation of the mix of analyte and reporter compound compared to 20 min incubation of the same mixture. Furthermore, no loss in the sensitivity was observed for the reduced incubation time as shown in FIG. 20. The low incubation time and quick measurement time (˜10 s) demonstrates the feasibility of the platform for rapid diagnostic tests.

### Example 6—Biosensor Assay—Incubation Time of Reporter Compound (INHIBIT/Blocking)

The biosensor was produced with the same immobilized basis compounds and antifouling reagents and was converted to an analyte specific biosensor as in example 3.

Here the incubation time of the reporter compound was varied from 1 minute to 20 minutes.

The results are shown in FIG. 21. The assay was demonstrated to work equally well after only 1 min incubation of reporter compound compared to 20 min incubation. Furthermore, no loss in the sensitivity was observed for the reduced incubation time.

The low incubation time and quick measurement time (˜10 s) demonstrates the feasibility of the platform for rapid diagnostic tests.

1. A method of detecting an analyte in a sample, the method comprising
providing an electrochemical sensor system comprising an electrochemical biosensor and an electrochemical reader, wherein the electrochemical biosensor comprises a measuring compartment comprising a working electrode (WE), wherein the providing of the electrochemical sensor system comprises providing the working electrode to comprise immobilized basis compounds, wherein each basis compound of said immobilized basis compounds comprises an electrode-attachment moiety and a primary region; and
providing a reporter reagent comprising reporter compounds, wherein each reporter compound of said reporter compounds comprises a capture-binding region and a reporter moiety;
wherein said basis compounds are selected from the group consisting of i) linker compounds wherein the primary region is the linker region, ii) direct capture compounds wherein the primary region is the capture region and iii) pre-hybridized compounds of linker compounds each comprising a linker region and capture compounds each comprising a linker-binding region and a capture region, wherein the linker binding region is hybridized with the linker region and the primary region is the capture region,
wherein, when the primary region is a linker region the method comprises providing a capture reagent comprising capture compounds, wherein each of said capture compound comprises a linker-binding region and a capture region, wherein said linker-binding region is adapted for hybridizing with said linker region, and
wherein each of said linker region, linker-binding region, capture region and capture-binding region comprises an oligonucleotide,
wherein said capture-binding region is adapted for hybridizing with said capture region and wherein one of said capture region and said capture-binding region comprises an aptamer for said analyte, and where at least part of said aptamer contributes to said hybridization of said capture region and said capture-binding region;
wherein the method comprises
applying a portion of said capture reagent to said measuring compartment and allowing said linker region to hybridize with said linker-binding region where said basis compounds are linker compounds,
subjecting a portion of said reporter reagent and said sample to said measuring compartment and
detecting said analyte in said sample, comprising reading out at least one electrical signal using said reader and quantitatively and/or qualitatively detecting said analyte based on said electrical signal.

2. The method of claim 1, wherein said basis compounds comprises linker compounds and wherein the method comprises providing the working electrode to comprise said immobilized basis compounds, comprising immobilizing said linker compounds via said electrode-attachment moiety to said working electrode and
wherein the method comprises
providing a capture reagent comprising capture compounds, wherein each capture compound of said capture compounds comprises a linker-binding region and a capture region, wherein said linker-binding region is adapted for hybridizing with said linker region, wherein said capture-binding region is adapted for hybridizing with said capture region and wherein one of said capture region and said capture-binding region comprises an aptamer for said analyte, and where at least part of said aptamer contributes to said hybridization of said capture region and said capture-binding region;
applying a portion of said capture reagent to said measuring compartment and allowing said linker region to hybridize with said linker-binding region;
subjecting a portion of said reporter reagent and said sample to said measuring compartment and
detecting said analyte, comprising reading out at least one electrical signal using said reader and quantitatively and/or qualitatively detecting said analyte based on said electrical signal.

3. The method of claim 1, wherein the providing of the electrochemical sensor system comprises providing the working electrode to comprise the immobilized basis compounds to comprise the primary region in the form of a capture region, wherein said immobilized basic compounds are selected from the group consisting of said direct capture compounds and said pre-hybridized compounds.
wherein said capture-binding region is adapted for hybridizing with said capture region and wherein one of said capture region and said capture-binding region comprises an aptamer for said analyte, and where at least part of said aptamer contributes to said hybridization of said capture region and said capture-binding region;
wherein the method comprises
subjecting a portion of said reporter reagent and said sample to said measuring compartment and
detecting said analyte, comprising reading out at least one electrical signal using said reader and quantitatively and/or qualitatively detecting said analyte based on said electrical signal.

4. The method of claim 1, wherein said oligonucleotide of at least one of the primary region, the capture region and the linker region is a PNA oligonucleotide.

5. The method of claim 1, wherein the method comprises providing the working electrode to comprise antifouling reagents immobilized to the working electrode, wherein the antifouling reagents comprises at least one of PEG poly(ethylene glycol) (PEG), zwitterions, alkanethiols, functionalized n-alkanethiols, proteins and/or any combinations comprising one or more of these reagents.

6. The method of claim 1, wherein the method comprises providing the working electrode to comprise antifouling reagents immobilized to the working electrode comprising providing a plurality of said basis compound comprises at least one antifouling moiety located between the electrode-attachment moiety and the primary region, wherein the antifouling moiety is selected from the group consisting of a zwitterionic moiety and a PEG (poly(ethylene glycol)) based moiety.

7. The method of claim 1, wherein method comprises subjecting said portion of reporter reagent and said sample to said measuring compartment simultaneously.

8. The method of claim 1, wherein said capture region comprises said aptamer for said analyte and wherein said step of subjecting of said reporter reagent and said sample to said measuring compartment comprises a competing step comprising adding said portion of reporter reagent and said sample simultaneously to said measuring compartment and allowing each of said capture region to hybridize to said capture-binding region of one of said reporter compounds of said portion of reporter reagent or to bind one optional analyte of said sample.

9. The method of claim 1, wherein said capture region comprises said aptamer for said analyte and wherein said step of subjecting of said portion of reporter reagent and said sample to said measuring compartment comprises a blocking step comprising adding said sample to said measuring compartment and allowing each of said capture regions to bind one optional analyte of said sample, followed by a step of adding said portion of reporter reagent to said measuring compartment allowing each of said capture regions that has not bound analyte to hybridize with said capture-binding region of one of said reporter compounds of said reporter reagent.

10. The method of claim 1, wherein said capture-binding region comprises said aptamer for said analyte, and wherein the step of subjection of said portion of reporter reagent to said sample and said sample to said measuring compartment comprises mixing said portion of reporter reagent with said sample and adding the mixture to said measuring compartment.

11. The method of claim 10, wherein said method comprises providing an incubation period of at least 1 second between the step of mixing said portion of reporter reagent with said sample and the step of adding the mixture to said measuring compartment and/or between adding the mixture to said measuring compartment and reading out the electrical signal.

12. The method of claim 1, wherein the method comprises subtracting background noise of the at least one electrical signal to obtain an at least one filtered electrical signal comprising fitting the least one electrical signal to a background correction algorithm, wherein the at least one electrical signal comprises at least one series of electrical signals comprising a plurality of measured data points and wherein the method comprises fitting the electrical signals to the background correction algorithm to obtain a corrected signal, wherein the background correction algorithm is selected from linear least squares fitting functions and non-linear least squares fitting functions such as exponentials or power-laws, optional applying initial start guess of at least one parameter, optionally determined directly from the electrical signal.

13. The method of claim 12, wherein the at least one filtered signal comprises a plurality of series of electrical signals, each series of electrical signals comprises a plurality of electrical signals selected from potentiometric signals, amperometric signals and/or impedometric signals,
wherein each of the series of electrical signals are determined as consecutive series of electrical signals and wherein the method comprises determining a peak height of each of the series of electrical signals to determine time dependent peak heights and determining a time-dependent change of peak height by
performing linear least squares fitting to one or more functions such as one or more polynomials; or
performing non-linear least squares fitting to one or more functions such as one or more exponential functions.

14. The method of claim 13, wherein the determination of the peak height of each of the series of electrical signals and determining the time-dependent change of peak height comprises determining a peak height slope between a peak height of one series of electrical signals and a next series of electrical signals and recalculating the peak height slope for each of the consecutive series of electrical signals until a stable peak height slope is achieved and deeming the stable peak height slope to be a plateau peak height slope.

15. The method of claim 14, wherein the method comprises determining a concentration of the analyte by correlating the plateau peak height slope to a calibration curve based on measurement on reference samples with known concentration of the analyte.

16. The method of claim 1, wherein the method comprises applying a filter element to cover at least the measuring compartment of the electrochemical biosensor, wherein the filter element comprises a porous filter material and wherein the filter element comprises said reporter compounds as dry reporter compounds and wherein the subjection of the reporter reagent to the measuring compartment comprises adding a liquid comprising at least one of the sample and a buffer to the filter element for resuspending the reporter compound and thereby subjecting of the reporter agent to the measuring compartment.

17. The method of claim 1, wherein said capture-binding region of said each reporter compound comprises an aptamer for said analyte, wherein said capture region is a PNA oligonucleotide, wherein the PNA oligonucleotide is adapted for hybridizing with at least a portion of said aptamer, and wherein the step of subjection of said portion of reporter reagent and said sample to said measuring compartment comprises subjecting said portion of reporter reagent and said sample to said measuring compartment simultaneously or adding said sample to said measuring compartment followed by adding said portion of reporter reagent to said measuring compartment.

18. An electrochemical biosensor for measuring an analyte in a sample, wherein the electrochemical biosensor comprises a measuring compartment for the sample to be analyzed, wherein said measuring compartment comprises a plurality of electrodes, wherein each electrode comprises an electrical lead, wherein the electrodes are electrically insulated from each other and, wherein the plurality of electrodes comprises at least one WE, wherein the working electrode comprises a plurality of immobilized basis compounds, wherein each of said basis compounds comprises an electrode-attachment moiety and a primary region,
wherein said basis compounds are selected from the group consisting of i) linker compounds wherein the primary region is a linker region, ii) direct capture compounds wherein the primary region is a capture region and iii) pre-hybridized compounds of linker compounds comprising a linker region and capture compounds each comprising a linker-binding region and a capture region, wherein the linker binding region is hybridized with the linker region and the primary region is the capture region.

19. The electrochemical biosensor of claim 18, wherein the electrochemical biosensor comprises a filter element covering at least the measuring compartment of the electrochemical bio sensor, wherein the filter element comprises a porous filter material, wherein the filter element comprises reporter compounds in the form of dry reporter compounds adapted for being resuspended and added to the measuring compartment by adding a liquid comprising at least one of the sample and a buffer to the filter element.

20. A method of detecting an analyte in a sample, the method comprising
providing an electrochemical biosensor comprises a measuring compartment for the sample to be analyzed, wherein said measuring compartment comprises a plurality of electrodes, wherein each electrode comprises an electrical lead, wherein the electrodes are electrically insulated from each other and, wherein the plurality of electrodes comprises at least one WE, wherein the working electrode comprises a plurality of immobilized linker compounds, wherein each of said linker compounds comprises an electrode-attachment moiety and a linker region;
preparing a capture regent comprising capture compounds each comprising a linker-binding region and a capture region, wherein the linker binding region is adapted for hybridizing with the linker region and the wherein the capture region is adapted hybridizing with the analyte;
preparing a reporter reagent comprising reporter compounds, wherein each reporter compound of said reporter compounds comprises a capture-binding region and a reporter moiety, wherein the capture-binding region comprises an aptamer for the analyte;
wherein the method comprises
applying a portion of said capture reagent to said measuring compartment and allowing said linker region to hybridize with said linker-binding,
subjecting a portion of said reporter reagent and said sample to said measuring compartment and
detecting said analyte, comprising reading out at least one electrical signal using said reader and quantitatively and/or qualitatively detecting said analyte based on said electrical signal.
